SEQUENCE LISTING (4 sequences)

SEQ ID NO: 1 - DNA, 20 bp, Artificial Sequence
  forward primer
agagctacgagctgcctgac

SEQ ID NO: 2 - DNA, 20 bp, Artificial Sequence
  reverse primer
agcactgtgttggcgtacag

SEQ ID NO: 3 - DNA, 23 bp, Artificial Sequence
  forward primer
atatgttctcctgcctactggaa

SEQ ID NO: 4 - DNA, 22 bp, Artificial Sequence
  reverse primer
gcccttcacgttattgcagatg

(19) United States
(12) Patent Application Publication
(10) Pub. No.: US 2022/0193047 A1
(43) Pub. Date: Jun. 23, 2022

(54) METHODS OF ENHANCING RADIOTHERAPY USING FERROPTOSIS INDUCERS AS RADIOSENSITIZERS

(71) Applicant: THE TRUSTEES OF COLUMBIA UNIVERSITY IN THE CITY OF NEW YORK, New York, NY (US)

(72) Inventors: Brent R. Stockwell, New York, NY (US); Ling Feng Ye, New York, NY (US); Simon Cheng, New York, NY (US)

(21) Appl. No.: 17/685,148

(22) Filed: Mar. 2, 2022

Related U.S. Application Data
(63) Continuation of application No. PCT/US2020/049859, filed on Sep. 9, 2020.
(60) Provisional application No. 62/900,254, filed on Sep. 13, 2019.

(51) Int. Cl. A61K 31/436; A61K 31/4545; A61K 31/517; A61K 31/553; A61K 31/704; C12Q 1/6886; A61P 35/00

(52) U.S. Cl. CPC A61K 31/436; A61K 31/4545; A61K 31/517; C12Q 2600/154; A61K 31/704; C12Q 1/6886; A61P 35/00; A61K 31/553

(57) ABSTRACT

The present disclosure provides, inter alia, methods for treating or ameliorating the effects of a cancer in a subject in need thereof by combining a radiosensitizer such as a ferroptosis inducer with radiation. Methods for identifying and treating a subject with a cancer that is resistant to radiotherapy, methods for enhancing the anti-tumor effect of radiation in a subject undergoing radiotherapy, and methods for enhancing the effect of radiation on a cancer cell are also provided.

### CROSS REFERENCE TO RELATED APPLICATIONS

The present application is a continuation of PCT international application no. PCT/US2020/049859, filed on Sep. 9, 2020, which claims benefit of U.S. Provisional Patent Application Ser. No. 62/900,254, filed on Sep. 13, 2019. The entire contents of the aforementioned applications are incorporated by reference as if recited in full herein.

### GOVERNMENT FUNDING

This invention was made with government support under grant no. CA209896, awarded by the National Institutes of Health. The government has certain rights in the invention.

### FIELD OF DISCLOSURE

The present disclosure provides, inter alia, methods of enhancing radiotherapy in a subject using ferroptosis inducers as radiosensitizers.

### INCORPORATION BY REFERENCE OF SEQUENCE LISTING

This application contains references to amino acids and/or nucleic acid sequences that have been filed concurrently herewith as sequence listing text file “CU19410-seq.txt”, file size of 2 KB, created on Sep. 12, 2019. The aforementioned sequence listing is hereby incorporated by reference in its entirety pursuant to 37 C.F.R. § 1.52(e)(5).

### BACKGROUND OF THE DISCLOSURE

Radiation therapy is one of the most important therapeutic modalities in the treatment of cancer, which provides both curative and palliative strategies for disease management (Delaney et al., 2005). DNA damage is thought to be the principal target of radiation, and its extent and repair are the most crucial factors determining intrinsic tumor cell death from radiation (Morgan and Lawrence, 2015). While radiation provides targeted local control of malignant lesions, the addition of systemic treatments is often required to provide radiosensitizing effects to tumors, as well as to manage undetected distant disease. Thus, the combination of chemotherapy and radiation has become more common over the past 30 years (Souhami and Tobias, 2003). However, tumor control still remains poor with combination chemoradiation therapy in many locally advanced cancers, such as sarcomas, gliomas and non-small cell lung cancers, which are historically considered radioresistant (Gerszten et al., 2009; Tang et al., 2017).

Accordingly, there is a need for methods to overcome radioresistance and enhance the anti-tumor effect of radiotherapy. This disclosure is directed to meeting these and other needs.

### SUMMARY OF THE DISCLOSURE

Although radiation is widely used to treat cancers, resistance mechanisms often develop and involve activation of DNA repair and inhibition of apoptosis. Therefore, chemicals that exploit alternative cell death pathways to selectively sensitize cancer cells to radiation are valuable. The present disclosure provides that ferroptosis, a form of non-apoptotic cell death driven by lipid peroxidation, is partly responsible for radiation-induced cancer cell death. Moreover, small molecules activating ferroptosis synergize with radiation to induce cell death in several cancer types by enhancing lipid peroxidation, but do not increase DNA damage or apoptosis activation. Ferroptosis inducers synergized with cytoplasmic irradiation, but not nuclear irradiation. Finally, administration of ferroptosis inducers enhanced the anti-tumor effect of radiation in a murine xenograft model, and in human patient-derived models of lung adenocarcinoma and glioma. These results suggest that ferroptosis inducers may be effective radiosensitizers that can expand the efficacy and range of indications for radiation therapy.

Accordingly, one embodiment of the present disclosure is a method for treating or ameliorating the effects of a cancer in a subject in need thereof, comprising co-administering to the subject i) a therapeutically effective amount of an agent that induces cell death and ii) a therapeutically effective amount of radiation.

Another embodiment of the present disclosure is a method for identifying and treating a subject with a cancer that is resistant to radiotherapy, comprising: a) administering radiotherapy to the subject; b) obtaining a biological sample from the subject; c) determining a SLC7A11 RNA expression level in the sample and comparing it to a predetermined reference; d) identifying the subject as having a cancer that is resistant to radiotherapy, if the SLC7A11 RNA expression level determined in step (c) is significantly higher than the reference; and e) treating the subject identified in step (d) as having a cancer that is resistant to radiotherapy by administering to the subject an effective amount of a radiosensitizer.

Another embodiment of the present disclosure is a method for identifying and treating a subject with a cancer that is resistant to radiotherapy, comprising: a) administering radiotherapy to the subject; b) obtaining a biological sample from the subject; c) determining a SLC7A11 DNA methylation level in the sample and comparing it to a predetermined reference; d) identifying the subject as having a cancer that is resistant to radiotherapy, if the SLC7A11 DNA methylation level determined in step (c) is significantly lower than the reference; and e) treating the subject identified in step (d) as having a cancer that is resistant to radiotherapy by administering to the subject an effective amount of a radiosensitizer.

A further embodiment of the present disclosure is a method for enhancing the anti-tumor effect of radiation in a subject undergoing radiotherapy, comprising administering to the subject a therapeutically effective amount of a ferroptosis inducer.

Still another embodiment of the present disclosure is a method for enhancing the effect of radiation on a cancer cell, comprising contacting the cell with an effective amount of a ferroptosis inducer during radiation treatment.

### BRIEF DESCRIPTION OF THE DRAWINGS

The application file contains at least one drawing executed in color. Copies of this patent application with color drawing(s) will be provided by the Office upon request and payment of the necessary fee.

FIGS. 1A-1H show that IKE and RSL3 increase radiation sensitivity in cancer cell lines through lipid peroxidation. Data are plotted as mean±SEM; n=3 side-by-side experiments for FIGS. 1A, 1C, 1D, 1E, 1F, 1G, and 1H. Three biologically independent experiments were performed with similar results.

FIG. 1A shows the dose response of HT-1080 cells treated with DMSO, 300 nM IKE, or 20 nM RSL3 to radiation measured by clonogenic assays. ** represents p<0.01.

FIG. 1B shows the coefficients of interaction between IKE (top) or RSL3 (bottom) and radiation observed for 5 tested cancer cell lines measured by clonogenic assays.

FIG. 1C shows the dose response of HT-1080 cells treated with DMSO, 2 μM ferrostatin-1, or 25 μM Z-VAD-FMK to radiation measured by clonogenic assays. * represents p<0.05, n.s. represents p>0.05.

FIG. 1D shows the cell viability of HT-1080 cells treated with DMSO, 2 μM ferrostatin-1, 100 μM deferoxamine, 25 μM Z-VAD-FMK, 10 μM necrostatin-1S, or 5 mM 3-methyladenine and co-treated with 0 or 4 Gy radiation for 24 hours measured by Cell Titer Glo. Data normalized to 0 Gy unirradiated controls for each treatment group. ** represents p<0.01, * represents p<0.05, n.s. represents p>0.05. p values calculated with respect to cells treated with DMSO and 4 Gy IR.

FIG. 1E shows the dose response of HT-1080 cells treated with DMSO, 300 nM IKE, 2 μM ferrostatin-1, or 300 nM IKE and 2 μM ferrostatin-1 measured by clonogenic assays. ** represents p<0.01. Significance is calculated between the group treated with ferroptosis inducer and the group co-treated with ferroptosis inducer and ferroptosis inhibitor in E), F), G) and H).

FIG. 1F shows the dose response of HT-1080 cells treated with DMSO, 20 nM RSL3, 2 μM ferrostatin-1, or 20 nM RSL3 and 2 μM ferrostatin-1 measured by clonogenic assays. ***represents p<0.001.

FIG. 1G shows the dose response of HT-1080 cells treated with DMSO, 300 nM IKE, 25 μM Trolox, or 300 nM IKE and 25 μM Trolox measured by clonogenic assays. ** represents p<0.01.

FIG. 1H shows the dose response of HT-1080 cells treated with DMSO, 20 nM RSL3, 25 μM Trolox, or 20 nM RSL3 and 25 μM Trolox measured by clonogenic assays. *** represents p<0.001.

FIGS. 2A-2E show that markers of ferroptosis are elevated in HT-1080 cells treated with radiation. Data are plotted as mean±SEM; n=3 technical replicates for FIGS. 2A, 2B, 2D and 2E. Three biologically independent experiments were performed with similar results.

FIG. 2A shows the PTGS2 mRNA fold change measured by RT-qPCR in HT-1080 cells treated with DMSO, 100 nM RSL3, or 10 μM ferrostatin-1 and co-treated with 0 or 6 Gy radiation for 24 hours. **** represents p<0.0001.

FIG. 2B shows the MDA levels measured using the TBARS assay in HT-1080 cells treated with DMSO, 1 μM IKE, or 10 μM ferrostatin-1 and co-treated with 0 or 6 Gy radiation for 24 hours. *** represents p<0.001, ** represents p<0.01, * represents p<0.05.

FIG. 2C shows representative histograms of HT-1080 cells treated with DMSO, 1 μM IKE, or 1 μM IKE+10 μM ferrostatin-1 and co-treated with 0 or 6 Gy radiation for 24 hours and stained with C-11 BODIPY measured by flow cytometry. Horizontal bars indicate C-11 BODIPY-positive cell populations.

FIG. 2D shows C11-BODIPY staining of HT-1080 cells treated with ferroptosis modulators and co-treated with 0 or 6 Gy radiation measured by flow cytometry. ** represents p<0.01.

FIG. 2E shows that reduced glutathione (GSH) level is detected in HT-1080 cells treated with DMSO or 2 μM IKE and co-treated with 0, 2 or 6 Gy radiation for 24 hours using a fluorometric assay. **** represents p<0.0001, ** represents p<0.01, * represents p<0.05, n.s. represents p>0.05.

FIGS. 3A-3D show that IKE and RSL3 enhance radiation-induced cell death in HT-1080 cells through mechanisms independent of DNA damage or apoptosis. Data are plotted as mean±SEM; n=20 cells counted for B) and n=10 cells counted for FIG. 3C. Three biologically independent experiments were performed with similar results for FIGS. 3B, 3C and 3D.

FIG. 3A shows representative images of γH2AX immunofluorescence staining in HT-1080 cells treated with DMSO, 10 μM IKE, 1 μM RSL3, or 10 μM ferrostatin-1 and co-treated with 0 or 6 Gy radiation for 30 minutes or 6 hours. Blue: DAPI; Yellow: γH2AX-FITC. Scale bar, 10 μm.

FIG. 3B shows quantification of γH2AX immunofluorescence staining in HT-1080 cells treated with DMSO, 10 μM IKE, 1 μM RSL3, or 10 μM ferrostatin-1 and co-treated with 0 or 6 Gy radiation for 30 minutes or 6 hours. **** represents p<0.0001, n.s. represents p>0.05.

FIG. 3C shows Quantification of percent tail DNA in HT-1080 cells using the comet assay. Cells were treated with DMSO, 10 μM IKE, 1 μM RSL3 or 10 μM ferrostatin-1 and co-treated with 0 or 6 Gy radiation for 30 minutes or 4 hours. Percent tail DNA was calculated by dividing the total fluorescence intensity of the tail area by the total fluorescence intensity of the comet. *** represents p<0.001, ** represents p<0.01, * represents p<0.05, n.s. represents p>0.05.

FIG. 3D shows Western blot of cleaved caspase-3 in HT-1080 cells treated with DMSO, 1 μM IKE, 50 nM RSL3 and co-treated with 0 or 6 Gy radiation for 24 hours. Cells treated with 500 nM staurosporine and 500 nM staurosporine+100 μM Z-VAD-FMK were included as positive and negative controls.

FIGS. 4A-4C show that untargeted lipidomic study reveals enhanced ferroptosis lipid signatures in cells co-treated with IKE and radiation in HT-1080 cells.

FIG. 4A shows principal component analysis of the extracted lipid features in samples treated with DMSO or 5 μM IKE for 12 hours, with or without 6 Gy radiation for 24 hours, in both positive and negative electrospray ionization modes.

FIG. 4B shows the fold change heatmap of significantly changed lipid features from both IKE treatment and radiation treatment determined by two-way ANOVA (FDR corrected p value <0.05). Blue indicates decreased abundance compared to DMSO-treated controls (fold changes between 0.3 and 0.8); white indicates no change (fold changes between 0.8 and 1.2); red indicates increased abundance (fold changes between 1.2 and 10). n=3 biologically independent samples. Abbreviations: FA, fatty acid; LysoPC, lysophosphatidylcholine; LysoPE, lysophosphatidylethanolamine; LysoPI, lysophosphatidylinositol; DAG, diacylglycerol.

FIG. 4C shows a proposed model of how oxidation of membrane polyunsaturated fatty acids by IKE and radiation cause elevated lysophospholipids and cell death. Abbreviations: PUFA, polyunsaturated fatty acid; PLA2, phospholipase A2; Lyso-PL, lysophospholipid.

FIGS. 5A-5E show that ferroptosis inducers synergize with cytoplasmic irradiation but not nuclear irradiation in HT-1080 cells. Data are plotted as mean±SEM; n=3 side-by-side experiments for FIGS. 5B and 5C and n=20 cells counted for FIGS. 5D and 5E. Three biologically independent experiments were performed with similar results.

FIG. 5A is a diagram of microbeam setup showing locations of beam spots targeting either the nucleus or cytoplasm.

FIG. 5B shows clonogenic cell survival of HT-1080 cells treated with nuclear radiation and IKE or RSL3. CDI values are indicated below data points according to the formula CDI=AB/(A×B), where AB is the surviving fraction of the combination treatment, and A and B are the surviving fractions of the individual treatments. CDI <1 indicates synergy, CDI=1 indicates additivity, and CDI >1 indicates antagonism.

FIG. 5C shows clonogenic cell survival of HT-1080 cells treated with cytoplasmic radiation and IKE or RSL3. CDI values are indicated above data points.

FIG. 5D shows immunofluorescence staining of γH2AX in untreated cells, and cells treated with 100 protons to the nucleus or 2000 protons to the cytoplasm for 30 minutes. Blue: DAPI; Yellow: γH2AX-FITC. **** represents p<0.0001, n.s. represents p>0.05. Scale bar, 10 μm.

FIG. 5E shows immunofluorescence staining of 4-HNE in untreated cells, and cells treated with 100 protons to the nucleus or 2000 protons to the cytoplasm for 2 hours. Blue: DAPI; Red: 4-HNE-Rhodamine Red. **** represents p<0.0001, n.s. represents p>0.05. Scale bar, 10 μm.

FIGS. 6A-6D show that IKE and sorafenib, combined with stereotactic radiation therapy, suppress tumor growth in a mouse xenograft model of sarcoma. Data are plotted as mean±SEM.

FIG. 6A shows the tumor volume ratio change in HT-1080 xenograft tumors treated with vehicle or 40 mg/kg IKE i.p. for 14 days and co-treated with 0 or 6 Gy radiation on days 2 and 4. n=7 or 8 mice per group. ** represents p<0.01, * represents p<0.05, n.s. represents p>0.05.

FIG. 6B shows immunofluorescence staining and quantification of MDA on paraffin-embedded tumor tissue sections measured by confocal microscopy. Blue: DAPI; Green: MDA-FITC. Scale bar, 50 μm. *** represents p<0.01, n.s. represents p>0.05. n=20 images with sections cut from four randomly chosen mice from each group, and five images captured from each section.

FIG. 6C shows the tumor volume ratio change in HT-1080 xenograft tumors treated with vehicle or 40 mg/kg sorafenib i.p. for 14 days and co-treated with 0 or 6 Gy radiation on days 1 and 3. n=4 or 5 mice per group. ** represents p<0.01, * represents p<0.05, n.s. represents p>0.05.

FIG. 6D shows that reduced glutathione (GSH) level is detected, in HT-1080 xenograft tumors treated with vehicle or 40 mg/kg sorafenib i.p. for 14 days and co-treated with 0 or 6 Gy radiation on days 1 and 3, using a fluorometric assay. **** represents p<0.0001, ** represents p<0.01, * represents p<0.05. n=3 tumor samples from different animals per group.

FIGS. 7A-7F show that SLC7A11 is a target for radiosensitization in human models of glioma and lung adenocarcinoma. Data are plotted as mean±SEM.

FIG. 7A shows Kaplan-Meier survival analysis of overall survival of TCGA glioma patients in quartile 1 (low) and quartile 4 (high) of SLC7A11 RNA expression (left) or DNA methylation (right).

FIG. 7B shows hazard ratios for disease-free survival between patients in quartile 1 (low) and quartile 4 (high) of SLC7A11 RNA expression or DNA methylation either in the case of radiation treatment, no radiation treatment, or all cases.

FIG. 7C shows representative histograms of a human diffuse astrocytoma slice culture sample treated with DMSO, 10 μM IKE, or 10 μM IKE+10 μM ferrostatin-1, co-treated with 0 or 2 Gy radiation for 24 hours, dissociated, stained with H2DCFDA, and measured by flow cytometry. Horizontal bars indicate H2DCFDA-positive cell populations.

FIG. 7D shows H2DCFDA staining of three human glioma slice culture samples treated with DMSO, 10 μM IKE, or 10 μM IKE+10 μM ferrostatin-1, co-treated with 0 or 2 Gy radiation for 24 hours, dissociated, stained with H2DCFDA, and measured by flow cytometry. * represents p<0.05. n=3 samples from distinct human glioma patients.

FIG. 7E shows the tumor volume ratio change in TM00219 patient-derived xenograft tumors treated with vehicle or 40 mg/kg IKE i.p. for 14 days and co-treated with 0 or 6 Gy radiation on day 1. n=5 or 6 mice per group. **** represents p<0.0001, ** represents p<0.01.

FIG. 7F shows the tumor volume ratio change in TM00219 patient-derived xenograft tumors treated with vehicle or 40 mg/kg sorafenib i.p. for 14 days and co-treated with 0 or 6 Gy radiation on day 1. n=5 or 6 mice per group. *** represents p<0.001, ** represents p<0.01.

FIGS. 8A-8E show the survival data of cell lines treated with cell death inducers. FIG. 8A shows the dose response of HT-1080 cells treated with DMSO, staurosporine, doxorubicin, rapamycin, or TNFα+Z-VAD-FMK+Birinapant to radiation measured by clonogenic assays. FIGS. 8B to 8E show the dose response of A549, PC9, SK-LMS-1, and U87 cells treated with DMSO, IKE, or RSL3 to radiation measured by clonogenic assays. CDI values are indicated next to data points according to the formula CDI=AB/(A×B), where AB is the surviving fraction of the combination treatment, and A and B are the surviving fractions of the individual treatments. Data are plotted as mean±SEM; n=3 for A) and n=2 for B) side-by-side replicates. Three biologically independent experiments were performed with similar results.

FIG. 9 shows clonogenic survival of HT-1080 cells treated with vehicle, N-acetylcysteine, or glutathione methyl ester to 0, 2, 4 and 6 Gy radiation. Data are plotted as mean±SEM; n=2 side-by-side replicates. The experiment was repeated twice with similar results.

FIG. 10 shows cell viability of HT-1080 cells treated with DMSO or Z-VAD-FMK and co-treated with 0 or 4 Gy radiation for 48, 72 or 96 hours measured by Cell Titer Glo. Data normalized to 0 Gy unirradiated controls for each group. Data are plotted as mean±SEM; n=3 side-by-side replicates. The experiment was repeated twice with similar results.

FIG. 11 shows the dose response of HT-1080 cells treated with nuclear (left) or cytoplasmic (right) microbeam radiation, measured by clonogenic assays. Data are plotted as mean±SEM; n=3 side-by-side replicates.

FIGS. 12A-12B show the mice weight measured by electronic balance over 14 days. Data are plotted as mean±SEM; n=7 or 8 mice per group for FIG. 12A, n=4 or 5 mice per group for FIG. 12B.

FIGS. 13A-13B show the effect of sorafenib and radiation on survival and intracellular GSH of HT-1080 cells. FIG. 13A shows the dose response of HT-1080 cells treated with DMSO, sorafenib, ferrostatin-1, or sorafenib+ferrostatin-1 to radiation measured by clonogenic assays. CDI values are indicated next to data points according to the formula CDI=AB/(A×B), where AB is the surviving fraction of the combination treatment, and A and B are the surviving fractions of the individual treatments. FIG. 13B shows the reduced glutathione (GSH) level detected in HT-1080 cells treated with DMSO or sorafenib and co-treated with 0 or 6 Gy radiation for 24 hours using a fluorometric assay. **** represents p<0.0001, *** represents p<0.001, * represents p<0.05. Data are plotted as mean±SEM. n=3 side-by-side replicates.

FIG. 14 shows Kaplan-Meier survival analysis of disease-free survival of TCGA glioma patients in quartile 1 (low) and quartile 4 (high) of SLC7A11 RNA expression (top panel) or DNA methylation (bottom panel).

### DETAILED DESCRIPTION OF THE DISCLOSURE

Radiation resistance mechanisms often involve activation of DNA repair pathways and inhibition of apoptosis (Goldstein and Kastan, 2015; Kim et al., 2015; Willers et al., 2013). At the same time, alternative radiation-induced cell death pathways, such as necroptosis and autophagy, have been reported (Chaurasia et al., 2016; Nehs et al., 2011). If activated, these mechanisms may offer strategies for treating otherwise radioresistant tumors.

In addition to DNA damage, radiation also generates reactive oxygen species, which results in oxidation of other biomolecules, such as lipid oxidation (Walden et al., 1988). While this effect has largely remained unexplored, a phospholipid-peroxidation-driven form of regulated cell death, ferroptosis, has recently been identified and is of growing importance in a variety of biological and diseases processes (Dixon et al., 2012). Ferroptosis is induced when phospholipid-PUFA peroxidation overwhelms cellular defense systems, such as the capacity of the glutathione phospholipid peroxidase GPX4 (Stockwell et al., 2017). Numerous cancer cell lines, such as sarcomas, renal cell carcinoma, and diffuse large B-cell lymphomas, have been found to be particularly sensitive to ferroptosis (Yang et al., 2014; Yang and Stockwell, 2016); some of these cell lines are also sensitive in the context of xenograft tumor models (Yang et al., 2014; Zhang et al., 2019). These data suggest the hypothesis that radiation's anti-tumor efficacy may in some contexts be driven by triggering ferroptosis, and that ferroptosis inducers may be effective radiosensitizers.

Accordingly, one embodiment of the present disclosure is a method for treating or ameliorating the effects of a cancer in a subject in need thereof, comprising co-administering to the subject i) a therapeutically effective amount of an agent that induces cell death and ii) a therapeutically effective amount of radiation.

In some embodiments, the cell death is selected from apoptosis, autophagy, necroptosis and ferroptosis. In some embodiments, the cell death is ferroptosis. As used herein, “ferroptosis” means regulated cell death that is iron-dependent. Ferroptosis is characterized by the overwhelming, iron-dependent accumulation of lethal lipid reactive oxygen species. (Dixon et al., 2012) Ferroptosis is distinct from apoptosis, necrosis, and autophagy. (Id.) Assays for ferroptosis are as disclosed herein, for instance, in the Examples section.

In some embodiments, the agent is a ferroptosis inducer. Non-limiting examples of a ferroptosis inducer include erastin, imidazole ketone erastin (IKE), piperazine erastin (PE), sulfasalazine, sorafenib, Ras Synthetic Lethal 3 (RSL3), ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof. In some embodiments, the agent is selected from IKE, RSL3, sorafenib, and combinations thereof. As used herein, the terms “induce”, “induction”, “inducer” and grammatical variations thereof mean to increase the occurrence of ferroptosis.

In some embodiments, the subject is a mammal. In some embodiments, the mammal is selected from the group consisting of humans, primates, farm animals, and domestic animals. In some embodiments, the subject is a human.

In some embodiments, the cancer is selected from the group consisting of sarcoma, renal cell carcinoma, diffuse large B-cell lymphoma, fibrosarcoma, glioma, uterine sarcoma, primary glioblastoma, lung cancer, non-small cell lung cancer, colorectal cancer, melanoma, prostate cancer, pancreatic cancer, brain cancer, breast cancer, colon cancer, liver cancer, leiomyosarcoma, lung adenocarcinoma, and hepatocyte-derived carcinoma. In some embodiments, the cancer is resistant to radiation.

In some embodiments, the co-administration of the agent and radiation provides a synergistic effect compared to administration of either the agent or radiation alone. As used herein, “co-administration” means administering the agent before, during and/or after radiation treatment. As used herein, “synergistic” means more than additive. Synergistic effects may be measured by various assays known in the art, including but not limited to those disclosed herein.

Another embodiment of the present disclosure is a method for identifying and treating a subject with a cancer that is resistant to radiotherapy, comprising: a) administering radiotherapy to the subject; b) obtaining a biological sample from the subject; c) determining a SLC7A11 RNA expression level in the sample and comparing it to a predetermined reference; d) identifying the subject as having a cancer that is resistant to radiotherapy, if the SLC7A11 RNA expression level determined in step (c) is significantly higher than the reference; and e) treating the subject identified in step (d) as having a cancer that is resistant to radiotherapy by administering to the subject an effective amount of a radiosensitizer.

Another embodiment of the present disclosure is a method for identifying and treating a subject with a cancer that is resistant to radiotherapy, comprising: a) administering radiotherapy to the subject; b) obtaining a biological sample from the subject; c) determining a SLC7A11 DNA methylation level in the sample and comparing it to a predetermined reference; d) identifying the subject as having a cancer that is resistant to radiotherapy, if the SLC7A11 DNA methylation level determined in step (c) is significantly lower than the reference; and e) treating the subject identified in step (d) as having a cancer that is resistant to radiotherapy by administering to the subject an effective amount of a radiosensitizer.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, the terms “radiosensitizer”, “radiation sensitizer”, “radio-enhancer” and grammatical variations thereof refer to an agent that makes tumor cells more sensitive to radiation therapy. In some embodiments, the radiosensitizer is a ferroptosis inducer selected from the group consisting of erastin, imidazole ketone erastin (IKE), piperazine erastin (PE), sulfasalazine, sorafenib, Ras Synthetic Lethal 3 (RSL3), ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof. In some embodiments, the radiosensitizer is selected from IKE, RSL3, sorafenib, and combinations thereof. In some embodiments, the radiosensitizer is administered before, during and/or after radiotherapy.

In some embodiments, the cancer is selected from the group consisting of sarcoma, renal cell carcinoma, diffuse large B-cell lymphoma, fibrosarcoma, glioma, uterine sarcoma, primary glioblastoma, lung cancer, non-small cell lung cancer, colorectal cancer, melanoma, prostate cancer, pancreatic cancer, brain cancer, breast cancer, colon cancer, liver cancer, leiomyosarcoma, lung adenocarcinoma, and hepatocyte-derived carcinoma. In some embodiments, the cancer is a glioma.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments, the subject is a human.

A further embodiment of the present disclosure is a method for enhancing the anti-tumor effect of radiation in a subject undergoing radiotherapy, comprising administering to the subject a therapeutically effective amount of a ferroptosis inducer.

Still another embodiment of the present disclosure is a method for enhancing the effect of radiation on a cancer cell, comprising contacting the cell with an effective amount of a ferroptosis inducer during radiation treatment.

As used herein, “contacting” means bringing the compound and optionally one or more additional therapeutic agents into close proximity to the sample such as cells in need of such induction. This may be accomplished using conventional techniques of drug delivery to the subject or in the in vitro situation by, e.g., providing the compound and optionally other therapeutic agents to a culture media in which the cells are located. In some embodiments, the ferroptosis inducer is selected from the group consisting of erastin, imidazole ketone erastin (IKE), piperazine erastin (PE), sulfasalazine, sorafenib, Ras Synthetic Lethal 3 (RSL3), ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof. In some embodiments, the ferroptosis inducer is selected from IKE, RSL3, sorafenib, and combinations thereof.

In some embodiments, the cell is a mammalian cell. Preferably, the mammalian cell is obtained from a mammal selected from the group consisting of humans, primates, farm animals, and domestic animals. More preferably, the mammalian cell is a human cancer cell. In some embodiments, the cancer cell is obtained from a cancer selected from the group consisting of sarcoma, renal cell carcinoma, diffuse large B-cell lymphoma, fibrosarcoma, glioma, uterine sarcoma, primary glioblastoma, lung cancer, non-small cell lung cancer, colorectal cancer, melanoma, prostate cancer, pancreatic cancer, brain cancer, breast cancer, colon cancer, liver cancer, leiomyosarcoma, lung adenocarcinoma, and hepatocyte-derived carcinoma. In some embodiments, the cancer cell is obtained from a glioma.

The following examples are provided to further illustrate the methods of the present disclosure. These examples are illustrative only and are not intended to limit the scope of the disclosure in any way.

### EXAMPLES

### Example 1

### Methods and Materials

KEY RESOURCES TABLE

| | | |
|---|---|---|
| REAGENT or RESOURCE | SOURCE | IDENTIFIER |

Antibodies

| | | |
|---|---|---|
| Anti-phospho histone-H2AX antibody | Millipore | 05-636 |
| Fluorescein (FITC) AffiniPure | Jackson | 115-095-003 |
| Goat Anti-Mouse IgG | ImmunoResearch | |
| (H + L) | | |
| Anti-cleaved caspase 3 antibody | Cell Signaling | 9661 |
| | Technology | |
| Anti-beta actin antibody | Sigma-Aldrich | A5316 |
| Peroxidase AffiniPure Goat | Jackson | 111-035-144 |
| Anti-Rabbit IgG | ImmunoResearch | |
| (H + L) | | |
| Anti-4 Hydroxynonenal antibody | Abcam | ab46545 |
| Rhodamine Red ™ -X (RRX) | Jackson | 111-295-144 |
| AffiniPure Goat Anti-Rabbit IgG | ImmunoResearch | |
| (H + L) | | |
| Anti-dihydropyridine-MDA-lysine | Reference | N/A |
| adduct mouse mAb 1F83 | (Yamada et al., | |
| | 2001) | |
| Goat Anti-Mouse IgG H&L (FITC) | Abcam | ab6785 |

Chemicals, Peptides, and Recombinant Proteins

| | | |
|---|---|---|
| Imidazole ketone erastin (IKE) | Reference | N/A |
| | (Larraufie et al., | |
| | 2015) | |
| Ras-synthetic lethal 3 (RSL3) | Reference (Yang | N/A |
| | et al., 2014) | |
| Ferrostatin-1 (Fer-1) | Reference (Skouta | N/A |
| | et al., 2014) | |
| Deferoxamine | Sigma-Aldrich | D9533 |
| Z-VAD-FMK | Selleck Chemicals | S7023 |
| Necrostatin-1S | Abcam | ab221984 |
| 3-Methyladenine | Sigma-Aldrich | M9281 |
| Trolox | Sigma-Aldrich | 238813 |
| Staurosporine | Selleck Chemicals | S1421 |
| Doxorubicin | ApexBio | A3966 |
| Rapamycin | PeproTech | 5318893 |
| Recombinant human TNFα | ABM | Z100859 |
| Birinapant | BioVision | 2597-1 |

Critical Commercial Assays

| | | |
|---|---|---|
| GSH/GSSG Ratio Detection | Abcam | ab13881 |
| Assay Kit | | |
| TBARS Assay Kit | Cayman | 700870 |
| BODIPY 581/591 C11 | Thermo Fisher | D3861 |
| | Scientific | |
| CellTiter-Glo Luminescent Cell | Promega | G7573 |
| Viability Assay | | |
| RNAeasy extraction kit | QIAGEN | 74106 |
| QIAshredder | QIAGEN | 79656 |
| High Capacity cDNA Reverse | Thermo Fisher | 4368814 |
| Transcription Kit | Scientific | |
| Power SYBR Green PCR | Thermo Fisher | 4368702 |
| Master Mix | Scientific | |
| Software and Algorithms | | |
| Prism, Version 7.0 | GraphPad | https://www.graphpad.com/ |
| | Software | scientific-software/prism/ |
| MassLynx, Version 4.1 | Waters | http://www.waters.com/ |
| | | waters/en_US/MassLynx- |
| | | Mass-Spectrometry-Software-/ |
| XCMS package, Version 3.2.0 | Bioconductor | http://packages.renjin.org/package/ |
| | | org.renjin.bioconductor/xcms |

### Experimental Model and Subject

Cell lines and cell culture information is listed below. The HT-1080 (male), SK-LMS-1 (female), U87 (male), and A549 (male) were obtained from ATCC. PC9 (male) cells were obtained from Sigma-Aldrich. HT-1080 cells were cultured in DMEM with 10% fetal bovine serum, 1% penicillin-streptomycin and 1% non-essential amino acids. SK-LMS-1 cells were cultured in EMEM with 10% fetal bovine serum and 1% penicillin-streptomycin. U87 cells were cultured in in DMEM with 10% fetal bovine serum and 1% penicillin-streptomycin. A549 cells were cultured in DMEM with 10% fetal bovine serum and 1% penicillin-streptomycin. PC9 cells were cultured in RPMI with 10% fetal bovine serum and 1% penicillin-streptomycin. All cells were maintained in a humidified environment at 37° C. and 5% CO₂ in an incubator.

The animal models used in the present disclosure are listed below. All animal study protocols were approved by the Columbia University Institutional Animal Care and Use Committee (IACUC). Athymic nude mice (Charles River Laboratories, strain code 490) (male, 8 weeks of age) and NSG mice (The Jackson Laboratory, female, 8 weeks of age) were acclimated after shipping for >3 days before beginning experiments. Mice were fed a standard diet and maintained with no more than 5 mice per cage.

### Clonogenic Assays

500 cells were seeded per well in 6-well plates in their respective growth media and incubated overnight. The next day, cells were co-treated with DMSO or compounds and radiation using a Gammacell 40 Caesium-137 irradiator (Theratronics). Plates were monitored every day using a light microscope for formation of colonies in the DMSO-treated wells. When colonies of >50 cells are clearly visible, the growth medium was discarded from all plates. The cells were washed with PBS, then fixed and stained with crystal violet solution (0.05% crystal violet, 1% formaldehyde, 1% methanol in PBS). Colonies were then directly visualized and counted.

### Cell Titer Glo Assay

HT-1080 cells were plated at 1,000 cells per well in white 96-well plates (100 uL per well) in technical duplicates. The cells were then treated with vehicle (DMSO), Ferrostatin-1, DFO, Z-VAD-FMK or Necrostatin-1S and incubated overnight. After 24 h incubation, cells were treated with 0 Gy or 2 Gy radiation using a Gammacell 40 Caesium-137 irradiator (Theratronics) and incubated overnight. After another 24 h, 100 uL of 50% Cell Titer Glo (Promega) 50% cell culture medium was added to each well and incubated at room temperature with shaking for 15 min. Luminescence was measured using a Victor X5 plate reader (PerkinElmer). Experiments were performed three independent times with different passages for each cell line.

### RT-qPCR

RNA was extracted using the Qiashredder and QIAGEN RNeasy Mini kits (QIAGEN) according to the manufacturer's protocol. 2 μg total RNA for each sample was used as input for each reverse transcription reaction. Quantitative PCR reactions were performed using the Power SYBR Green PCR Master Mix (Thermo Fisher Scientific). Triplicate samples per condition were analyzed on an Applied Biosystems 7300 qPCR instrument using absolute quantification settings. Differences in mRNA levels compared to ACTB internal reference control were computed between control and experimental conditions using the ΔΔCt method. The primers used in this study are listed below.

ACTB forward:
(SEQ ID NO: 1)
5′-AGAGCTACGAGCTGCCTGAC-3′
ACTB reverse:
(SEQ ID NO: 2)
5′-AGCACTGTGTTGGCGTACAG-3′
PTGS2 forward:
(SEQ ID NO: 3)
5′-ATATGTTCTCCTGCCTACTGGAA-3′
PTGS2 reverse:
(SEQ ID NO: 4)
5′-GCCCTTCACGTTATTGCAGATG-3′

### TBARS Assay

2 million cells were seeded in T175 flasks. The next day, cells were treated with compounds and/or radiation to induce MDA formation and incubated overnight. 24 h later, the cells were harvested, counted, and collected at 300× g at 4° C. for 5 min. The cell pellet was resuspended in 100 μl of RIPA buffer and homogenized by pipetting. The whole lysate was used to determine MDA concentration in each sample. We used the TBARS assay kit (Cayman Chemical) and followed the product instructions.

### Lipid ROS Assay Using Flow Cytometry

0.5 million cells were seeded in 10 cm dishes. The next day, they were co-treated with ferroptosis-modulating compounds and radiation and returned to the incubator. On the following day, cells were harvested in 15 ml tubes and washed twice with PBS followed by re-suspending in 500 μl of PBS containing 10 μM of BODIPY-C11 dye (Thermo Fisher Scientific, cat #D3861), and incubated at 37° C. for 30 minutes. The cells were then collected at 300× g for 5 minutes, washed with PBS three times, and subjected to the flow cytometry analysis. C6 flow cytometry system (BD Accuri cytometers) was used for the flow cytometer analysis. A minimum of 10,000 cells were analyzed per condition.

### Reduced Glutathione Measurement

1 million cells were seeded on 10 cm dishes. The next day, cells were treated with compounds and/or radiation to induce GSH depletion and incubated overnight. 24 h later, the cells were harvested and counted. Five million live cells from each sample were transferred to new tubes, and centrifuged at 300×g at 4° C. for 5 min. The cell pellet was resuspended in 100 μl of RIPA buffer and homogenized by pipetting. The lysate was centrifuged at 16,700×g at 4° C. for 15 min, and cleared lysate was used to determine the amount of GSH in the sample. We used the GSH/GSSG ratio detection assay kit (Abcam, #ab138881) and followed the product instructions to determine GSH levels.

### Immunofluorescence Study and Quantification of Cells

10,000 cells were seeded on cover slips placed inside wells of 6-well plates. The next day, cells were treated with ferroptosis modulators and/or radiation, then returned to the incubator for 30 minutes or 6 hours. After the growth medium was removed, the cells were washed with PBS and fixed with 100 μl 4% paraformaldehyde per well for 10 minutes at room temperature. After three washes with PBS, cells were permeabilized with 100 μl Triton X-100 (0.1% v/v) per well for 10 minutes incubation at room temperature. Non-specific protein binding was blocked with 100 μl of BSA (1% v/v) per well for 20 minutes at room temperature. After removing excess BSA, 100 μl of primary mouse monoclonal anti-phospho histone-H2AX antibody (1:500) was added to each well for a 1-hour incubation at room temperature. Cells were washed with PBS and incubated with 100 μl of secondary antibody per well for 45 minutes at room temperature in the dark. Cells were washed with PBS and mounted on slides with one drop of Prolong anti-fade reagent with DAPI (Invitrogen) per coverslip. Slides were stored at 4° C. in the dark before analysis on a Zeiss LSM 700 confocal microscope with constant laser intensity for all analyzed samples.

### Comet Assay

100,000 HT-1080 cells were seeded per well in 6-well plates and incubated overnight. On the next day, the cells were treated with ferroptosis modulators and/or radiation, then returned to the incubator for 30 minutes or 4 hours before being harvested with trypsin and counted. The comet assay was performed using the CometAssay kit (Trevigen) following the alkaline comet assay product instructions. All images were captured on a Zeiss LSM 700 confocal microscope with constant laser intensity for all analyzed samples. The fluorescent signal of each comet was analyzed using NIH ImageJ software.

### Western Blot

1 million cells were seeded in 10 cm dishes and treated with compounds and/or radiation on the next day. After 24 hours, cells were harvested at 300× g at 4° C. for 5 min, resuspended in 50 μl of RIPA buffer and homogenized by pipetting. After quantification by Bradford, samples were mixed with 5×SDS loading buffer and separated by SDS-polyacrylamide gel electrophoresis. After transfer, membranes were blocked for 10 min in Tris-buffered saline (pH 7.4) with 1% Tween-20 (TBS-T) with 5% milk and incubated in primary antibody overnight at 4° C. Following 3× for 5 min washes in TBS-T, the membrane was incubated with secondary antibodies for 1 hr. The membrane was washed again in TBST 3× for 5 min prior to visualization using enhanced chemiluminescence (ECL Western Blotting Substrate, Pierce). Antibody for cleaved caspase 3 (Cell Signaling Technology, #9661) was used at 1:1000 and detected using a Peroxidase AffiniPure Goat Anti-Rabbit IgG (H+L) (Jackson ImmunoResearch, 111-295-144) at 1:5000 dilution.

### Mass Spectrometry-Based Untargeted Lipidomics

### Sample Preparation

Lipids were extracted from each sample as described previously (Zhang et al., 2019). 5 million cells treated with DMSO or 5 μM IKE for 12 hours, with or without 6 Gy radiation for 24 hours, were scraped and homogenized in 250 mL cold methanol containing 0.01% butylated hydroxyl toluene (BHT) with micro tip sonicator. Homogenized samples were transferred to fresh glass tubes containing 850 mL of cold methyl-tertbutyl ether (MTBE) and vortex-mixed for 30 sec. To enhance extraction efficiency of lipids, the samples were incubated for one hour at 4° C. on an orbital shaker. Afterwards, 200 mL of cold water was added to each sample, and incubated for 20 min on ice before centrifugation at 3,000 rpm for 20 min at 4° C. The organic layer was collected followed by drying under a gentle stream of nitrogen gas on ice and stored at −80° C. until UPLC-MS analysis. The protein pellet was used to measure protein concentration for normalization using Bio-Rad protein assay. The samples were re-constituted in a solution containing 2-propanol/acetonitrile/water (4:3:1, v/v/v) containing mixture of internal standard (PLASH® LIPIDOMIX® Mass Spec Standard, Avanti Polar Lipids, INC.) for further UPLC-MS analysis. A quality control (QC) sample was prepared by combining 40 mL of each sample to assess the reproducibility of the features through the runs.

### Ultra-High-Performance Liquid Chromatography Analysis

Chromatographic separation of extracted lipids was carried out at 55° C. on ACQUITY UPLC CSH C18 Column, (130 Å, 1.7 μm, 2.1 mm×100 mm) over a 20-min gradient elution. Mobile phase A consisted of ACN/water (60:40, v/v) and mobile phase B was 2-propanol/ACN/water (85:10:5, v/v/v) both containing 10 mM ammonium acetate and 0.1% acetic acid. After injection, the gradient was held at 40% mobile phase B for 2 min. At 2.1 min, it reached to 50% B, then increased to 70% B in 12 min. At 12.1 min, changed to 70% B and in 18 min increased to 90% B. The eluent composition returned to the initial condition in 1 min, and the column was re-equilibrated for an additional 1 min before the next injection was conducted. The flow rate was set to 400 mL/min and Injection volumes were 6 μL using the flow through needle mode in both positive and negative ionization modes. The QC sample was injected between the samples and at the end of the run to monitor the performance and the stability of the MS platform. This QC sample was also injected at least 5 times at the beginning of the UPLC/MS run, in order to condition the column.

### Mass Spectrometry Analysis

The Synapt G2 mass spectrometer (Waters, Manchester, U.K.) was operated in both positive and negative electrospray ionization (ESI) modes. For positive mode, a capillary voltage and sampling cone voltage of 3 kV and 32 V were used. The source and desolvation temperature were kept at 120° C. and 500° C., respectively. Nitrogen was used as desolvation gas with a flow rate of 900 L/hr. For negative mode, a capillary voltage of −2 kV and a cone voltage of 30 V were used. The source temperature was 120° C., and desolvation gas flow was set to 900 L/hr. Dependent on the ionization mode the protonated molecular ion of leucine encephalin ([M+H]⁺, m/z 556.2771) or the deprotonated molecular ion ([M−H]⁻, m/z 554.2615) was used as a lock mass for mass accuracy and reproducibility. Leucine enkephalin was introduced to the lock mass at a concentration of 2 ng/mL (50% ACN containing 0.1% formic acid), and a flow rate of 10 mL/min. The data was collected in duplicates in the centroid data independent (MS^{E}) mode over the mass range m/z 50 to 1600 Da with an acquisition time of 0.1 seconds per scan. The QC samples were also acquired in enhanced data independent ion mobility (IMS-MS^{E}) in both positive and negative modes for enhancing the structural assignment of lipid species. The ESI source settings were the same as described above. The traveling wave velocity was set to 650 m/s and wave height was 40 V. The helium gas flow in the helium cell region of the ion-mobility spectrometry (IMS) cell was set to 180 mL/min to reduce the internal energy of the ions and minimize fragmentation. Nitrogen as the drift gas was held at a flow rate of 90 mL/min in the IMS cell. The low collision energy was set to 4 eV, and high collision energy was ramping from 25 to 65 eV in the transfer region of the T-Wave device to induce fragmentation of mobility-separated precursor ions.

### Data Pre-Processing and Statistical Analysis

All raw data files were converted to netCDF format using DataBridge tool implemented in MassLynx software (Waters, version 4.1). Then, they were subjected to peak-picking, retention time alignment, and grouping using XCMS package (version 3.2.0) in R (version 3.5.1) environment. For the peak picking, the CentWave algorithm was used with the peak width window of 2-25 s. For peak grouping, bandwidth and m/z-width of 2 s and 0.01 Da were used, respectively. After retention time alignment and filling missing peaks, an output data frame was generated containing the list of time-aligned detected features (m/z and retention time) and the relative signal intensity (area of the chromatographic peak) in each sample. Technical variations such as noise were assessed and removed from extracted features' list based on the ratios of average relative signal intensities of the blanks to QC samples (blank/QC >1.5). Also, peaks with variations larger than 30% in QCs were eliminated. All the extracted features were normalized to measured protein concentrations measured by BCA assay. Statistical analysis was performed in R (version 3.5.1) environment. Group differences were calculated using two-way ANOVA (p<0.05) and false discovery rate of 1% to control for multiple comparisons.

### Structural Assignment of Identified Lipids

Structural elucidation and validation of significant lipid features were initially obtained by searching monoisotopic masses against the available online databases such as METLIN, Lipid MAPS, and HMDB with a mass tolerance of 5 ppm. Fragment ion information obtained by tandem MS (UPLC-HDMSE) was utilized for further structural elucidation of significantly changed lipid species. HDMSE data were processed using MS^{E} data viewer (Version 1.3, Waters Corp., MA, USA).

### Microbeam Irradiation and Clonogenic Assay

The charged particle single cell microbeam at Radiological Research Accelerator Facility (RARAF) at the Center for Radiological Research, Columbia University was used. 300-500 HT-1080 cells were plated on polypropylene film treated with 3.5 μg/cm² Cell-Tak adhesive (BD Biosciences). The next day, cells were stained with Hoechst 33342 for 30 min. Cells were imaged, and their center-of-gravity coordinates were registered to automatically locate them by the microbeam control program. For nuclear irradiation, protons (¹H+) were directed by a precision beam to the center of the nucleus. For cytoplasmic irradiation, protons were directed to two locations 7 μm away from the ends of the major axis of each nucleus, as previously described (Wu et al., 1999). In each case, the beam has an accuracy of ±0.2 μm with 95% efficiency. Post-irradiation, cells were trypsinized and re-plated at a density of 500 cells per well in 6-well plates for clonogenic assays.

### Immunofluorescence Study and Quantification of Microbeam-Irradiated Cells

Microbeam-treated cells were washed three times with PBS, fixed for 15 min at room temperature in 4% (w/v) paraformaldehyde in PBS and then washed in PBS. The fixed cells were permeabilized with 0.1% Triton X-100 for 10 min and washed three times with PBS. The cells were incubated for 1 h at room temperature in PBS containing 5% (v/v) goat serum and then incubated for 1 h in the same medium containing Anti-phospho histone-H2AX antibody (Millipore, 05-636, 1:500 dilution) or Anti-4 Hydroxynonenal antibody (Abcam, ab46545, 1:500 dilution). The cells were washed and bound primary antibodies were detected by the reaction with Fluorescein (FITC) AffiniPure Goat Anti-Mouse IgG (H+L) (Jackson ImmunoResearch, 115-095-003, 1:1000 dilution) or Rhodamine Red TM-X (RRX) AffiniPure Goat Anti-Rabbit IgG (H+L) Jackson ImmunoResearch, 111-295-144, 1:1000 dilution) for 1 h. Cells were thoroughly washed and the polypropylene layer was cut out and placed on a glass slide. A cover glass was placed on top of the polypropylene layer and mounted using ProLong Diamond antifade mountant with DAPI (ThermoFisher P36962). Samples were examined using a Zeiss LSM 700 confocal microscope with constant laser intensity for all analyzed samples. The intensity above threshold of the fluorescent signal of the bound antibodies was analyzed using NIH ImageJ software. Fluorescence intensity was obtained using the images generated by Image J software (NIH, Bethesda, Md., USA).

### HT-1080 Tumor Xenograft Study in Mice

Athymic nude mice (8 weeks; Charles River Laboratories) were injected with four million HT-1080 cells s.c. After ˜14 days, when the flank tumors had reached an average volume of approximately 100 cubic millimeters, mice were randomized into 4 groups (vehicle or IKE treatment+/−radiation). 300 μL of vehicle (65% D5W, 30% PEG-400, 5% Tween-80) or 40 mg/kg IKE was delivered i.p. after sterilizing the solutions using a 0.2-micron syringe filter. The injections were repeated daily for 14 days. On days 2 and 4 of vehicle or IKE injections, 0 or 6 Gy radiation was delivered to the tumors using the Small Animal Radiation Research Platform (SARRP). For the sorafenib experiment, the formulation used was 40 mg/kg sorafenib in 5% DMSO, 20% ethanol in water containing 30% w/v cyclodextrin. Other experimental details were identical to the IKE experiment, with the exception that radiation was delivered on days 1 and 3. Tumor size was measured daily using calipers and mouse weight was measured daily. The animal protocols containing all the procedures were approved by Columbia University's IACUC.

### Patient-Derived Xenograft Study in Mice

Patient-derived xenograft tumor-bearing NSG cohort mice (TM00219, LG1049F Lung, 6-8 weeks of age) were purchased from The Jackson Laboratory. when the flank tumors had reached an average volume of approximately 60 cubic millimeters, mice were randomized into 4 groups (vehicle or IKE treatment or sorafenib treatment+/−radiation). The experiment was conducted according to the same protocol as the HT-1080 tumor xenograft study, with the exception that a single dose of radiation (sham or 6 Gy) was delivered to the tumor on day 1 of the experiment. The animal protocols containing all the procedures were approved by Columbia University's IACUC.

### Immunohistochemistry Study and Quantification on Paraffin-Embedded Tissue Sections

Tumor tissue was fixed in 4% paraformaldehyde (PFA) for 24 h at 4° C. followed by washing three times with PBS. The samples were fixed in paraffin. Six series of 5 mm sections were obtained with a sliding microtome. The serial sections were then mounted on gelatin-coated slide. The paraffin-embedded tissue sections were deparaffinized with xylene three times, 5 min each, followed by rehydrating in 100%, 90%, 70%, and 50% ethanol, two washes 5 min each, then rinsed with distilled water. Antigen retrieval was performed in sodium citrate buffer, pH 6.0, 95-100° C. for 10 min. Sections were then rinsed in PBST, 2 min each. A hydrophobic barrier pen was used to draw a circle on each slide. The slides were permeabilized with PBS/0.4% Triton X-100 twice before non-specific-binding blocking by incubating the sections with 10% goat serum (ThermoFisher 50197Z) for 30 minutes at room temperature. The sections were incubated with mouse anti-MDA mAb 1F83 (1:500 dilution) overnight at 4° C. in humidified chambers. Sections were washed with PBST for twice before incubating with goat anti-mouse IgG H&L (FITC) (Abcam, ab6785, 1:1000 dilution) at room temperature for 1 h. Slides were then washed twice with PBST. ProLong Diamond antifade mountant with DAPI (ThermoFisher P36962) was added onto slides, which were then covered with the coverslips, sealed by clear fingernail polish and observed under confocal microscopy. All images were captured on a Zeiss LSM 700 confocal microscope with constant laser intensity for all analyzed samples. The intensity above threshold of the fluorescent signal of the bound antibodies was analyzed using NIH ImageJ software.

### TCGA Glioma Expression Analysis

The Human Cancer Genome Atlas (TCGA) glioma dataset was downloaded using the cBioportal for Cancer Genomics, selecting all primary tumors. Statistical analyses were carried out using the IBM SPSS Statistics software package (International Business Machines Corporation, Armonk, N.Y.). Tumors in Quartile 1 has the lowest 25 percentile of measured level, while tumors in Quartile 4 has the highest 25 percentile of measured level. Differences in survival times were determined using the Kaplan-Meier method and significance was determined using the log-rank test. Hazard ratios were determined using the Cox Proportional Hazards Ratios (HR) model. 95% confidence intervals are expressed next to corresponding hazard ratios. Tests with two-tailed P values <0.05 were considered statistically significant.

Generation of Acute Organotypic Slice Cultures from Human Surgical Specimens

Human surgical specimens were collected from Columbia University Medical Center operating theaters in accordance with Institutional Review Board protocols. Surgical specimens were deidentified, placed in a sterile 50 mL conical tube containing ice-cold sucrose solution (210 mM sucrose, 10 mM glucose, 2.5 mM KCl, 1.25 mM NaH₂PO₄, 0.5 mM CaCl₂), 7 mM MgCl₂ and 26 mM NaHCO₃). Tissue specimens were let rest in the sucrose solution for 20 minutes. Tissue specimens were then cut into 300 μm sections using a Mcllwain Tissue Chopper. Sections after cutting were let rest in the sucrose solution for 20 minutes. Then the tissue sections were transferred onto Millicell cell culture inserts (0.4 μM, 30 mm diameter) and placed in 6-well plates containing 1.5 mL of media. The media consisted of DMEM/F12 plus N−2 Supplement. For drug treatment conditions, IKE (10 μM) and ferrostatin-1 (10 μM) were added to the media before tissue sections were plated. 24 hours after cells were plated in normal media or drug containing media, sections were treated with 2 Gy radiation.

### Dissociation of Slice Cultures for Flow Cytometric Analysis of Reactive Oxygen Species

Tissue sections were dissociated using Carica Papain extract diluted 1:20 in PBS. A final concentration of 1N NaOH, and 0.02 g of L-cysteine per 10 mL were added and then the enzymatic solution was filtered using a 0.4 μM filter. Sections were added to 1 mL of this enzymatic digestion solution in a 15 mL conical and incubated with shaking in a 37 C water bath for 30 minutes. Sections were spun down at 400 g×5 minutes. Papain was aspirated, and sections were re-suspended in 1 mL PBS and triturated with a P-1000 for 1 minute. 3 mL of PBS were added on top to further dilute the Papain solution. The dissociated solution was spun down at 400 g×5 minutes. Sections were re-suspended in 1 mL PBS, triturated with a glass-tip Pasteur pipette 10 times. 1 mL of 30% Sucrose solution in PBS was added. The section suspension was spun at 1000 g×5 minutes. The top 1.5 mL was removed. 500 μL was added to the 500 μL of the section suspension. This 1 mL of a single-cell suspension was transferred to an Eppendorf tube. Calcein Blue (final concentration 5 μM) and H₂DCFDA (final concentration 10 μM) were added to the single-cell suspensions and incubated in a water bath at 37 C for 10 minutes. Suspensions were spun down at 500 g×5 minutes and resuspended in PBS and taken for flow cytometric analysis on a LSRIII Fortessa machine.

### Quantification and Statistical Analysis

T-test, one-way ANOVA, and two-way ANOVA were performed in the R environment and GraphPad Prism7 with significance and confidence level 0.05 (95% confidence interval).

### Example 2

### IKE and RSL3 Synergize with Radiation to Promote Clonogenic Ferroptotic Cell Death in Cell Lines of Multiple Tumor Types

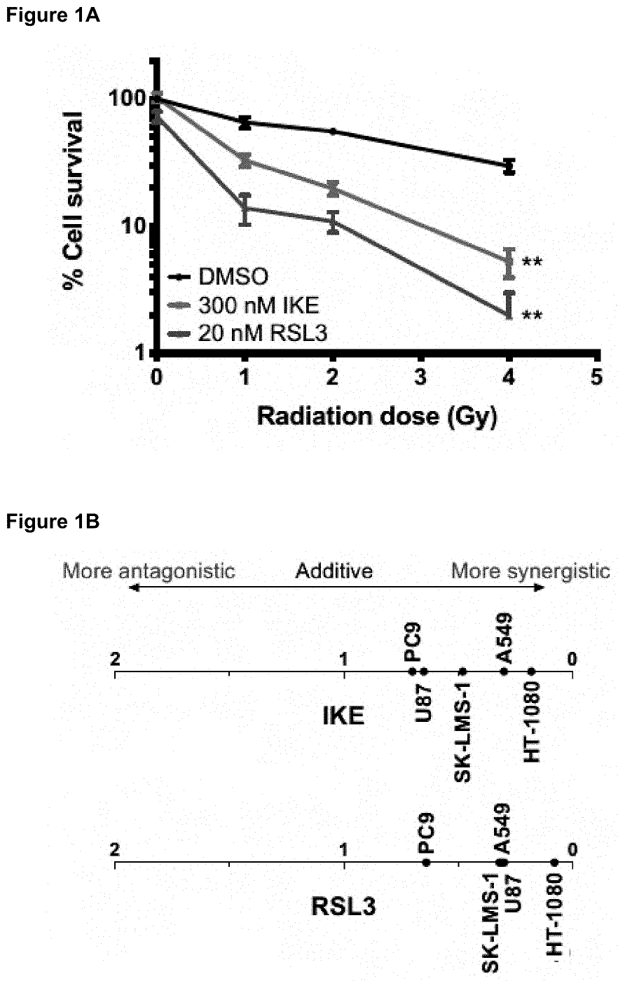

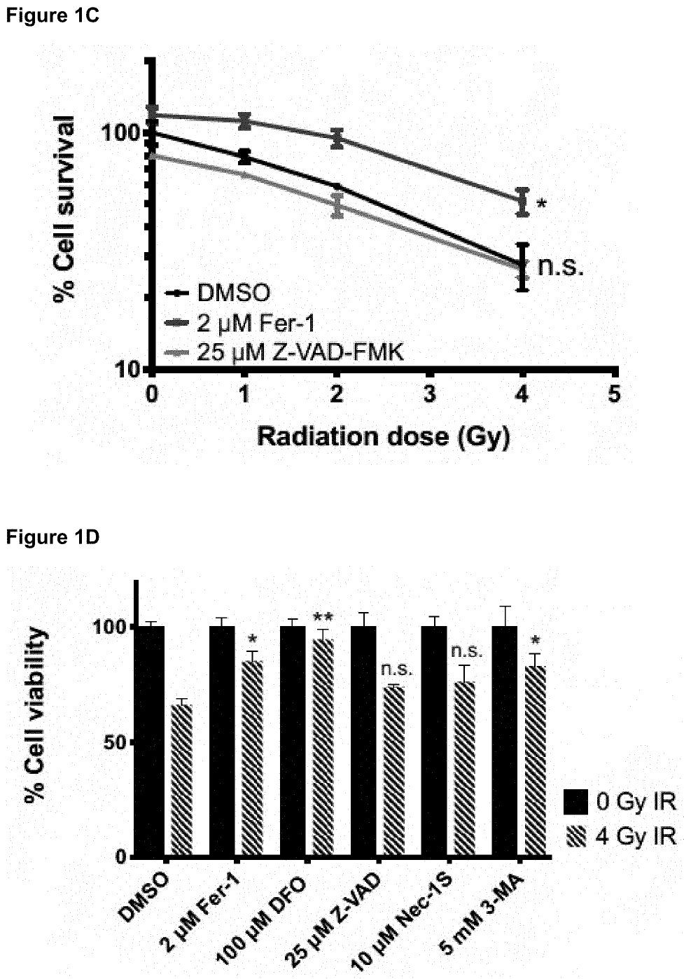

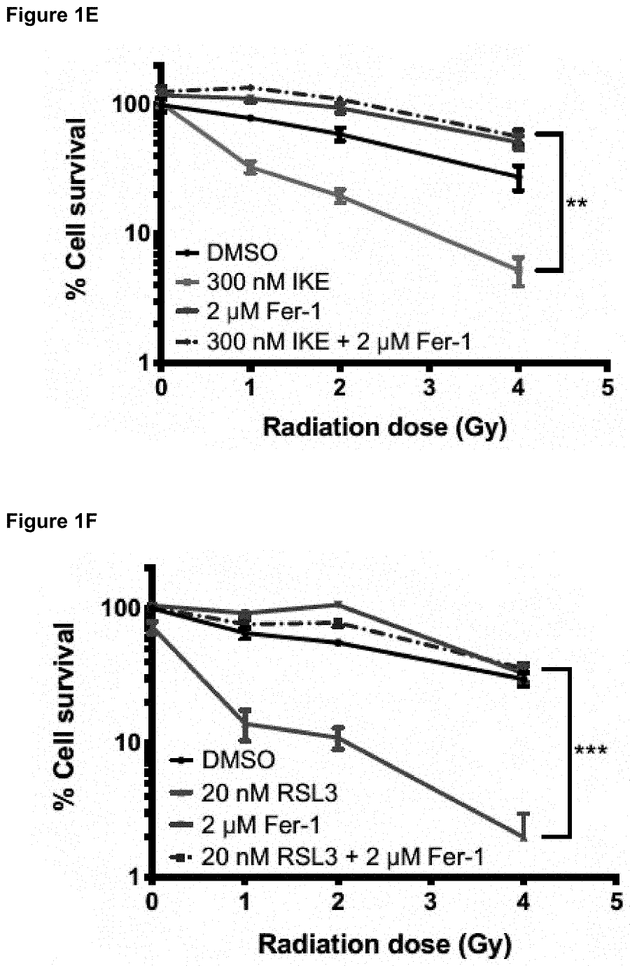

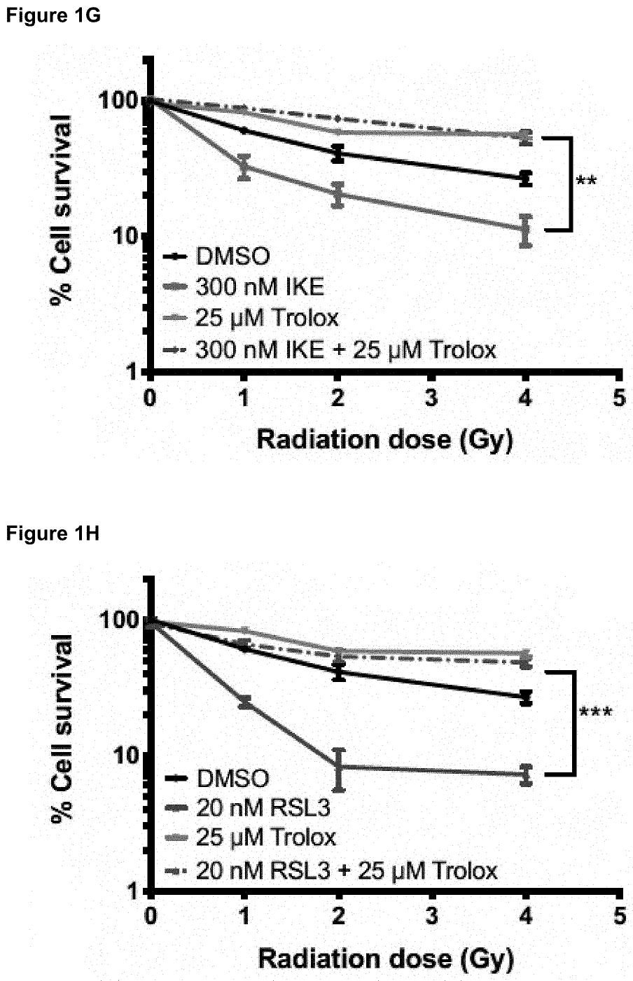

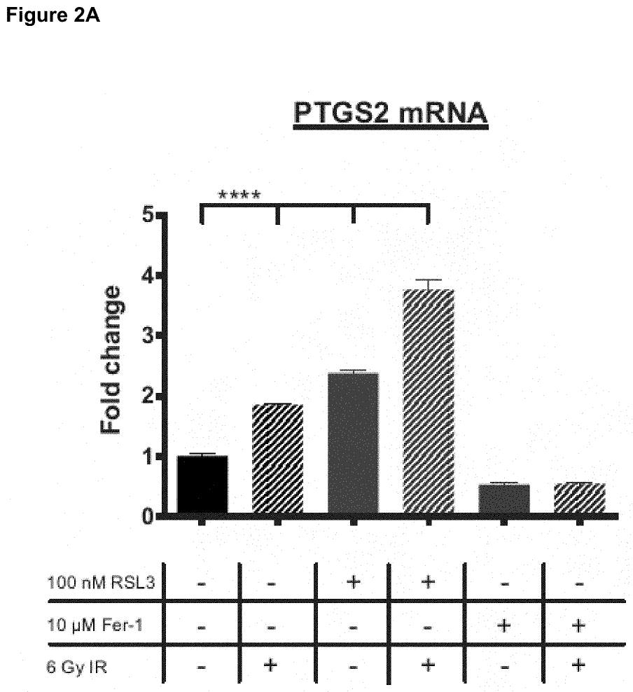

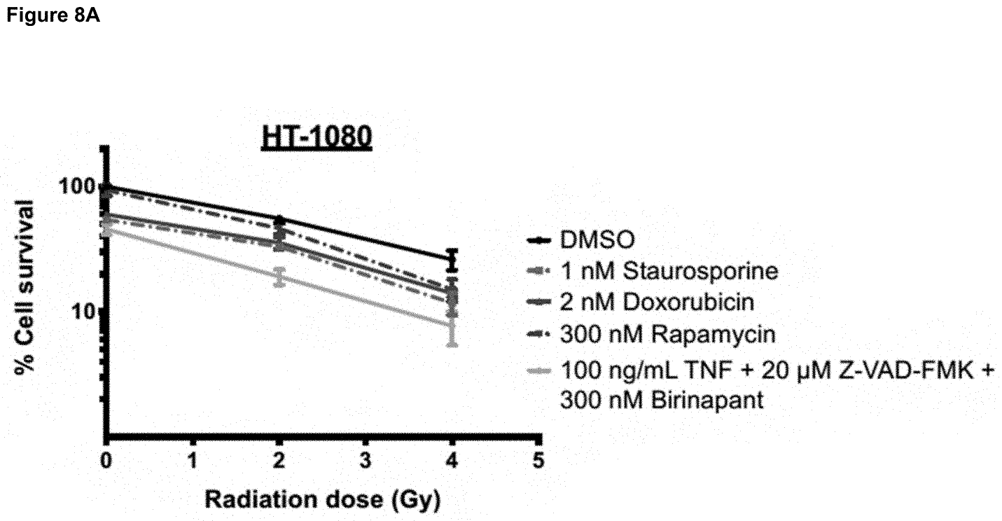

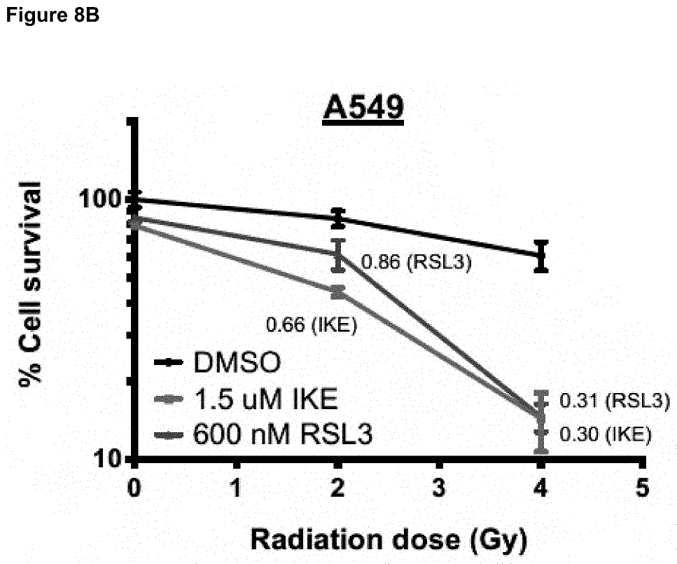

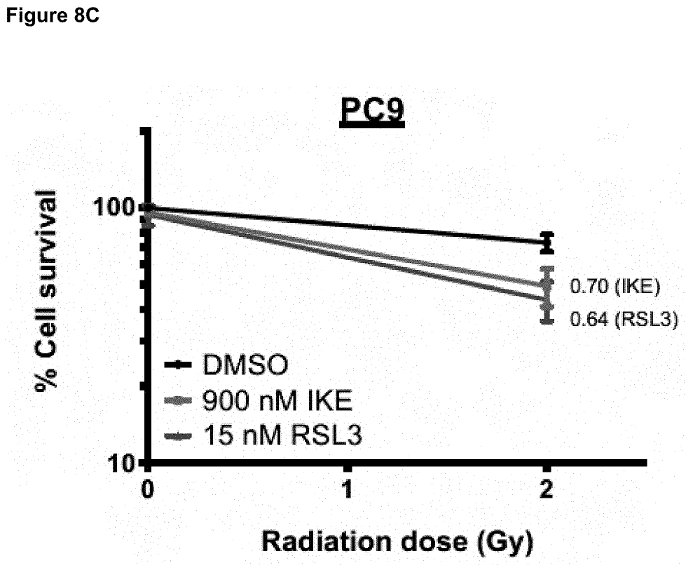

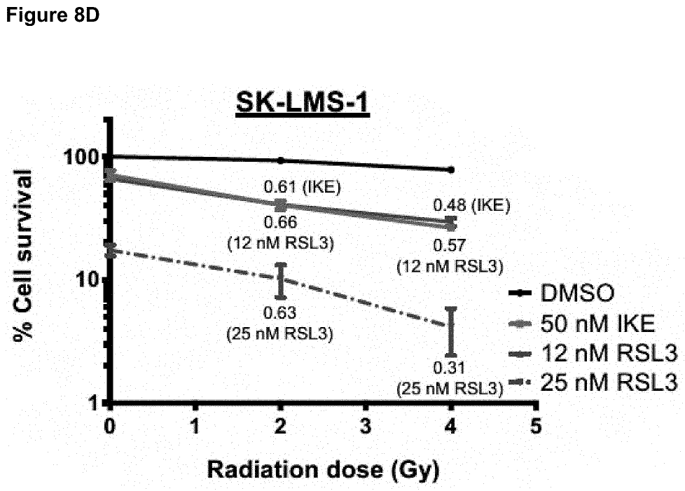

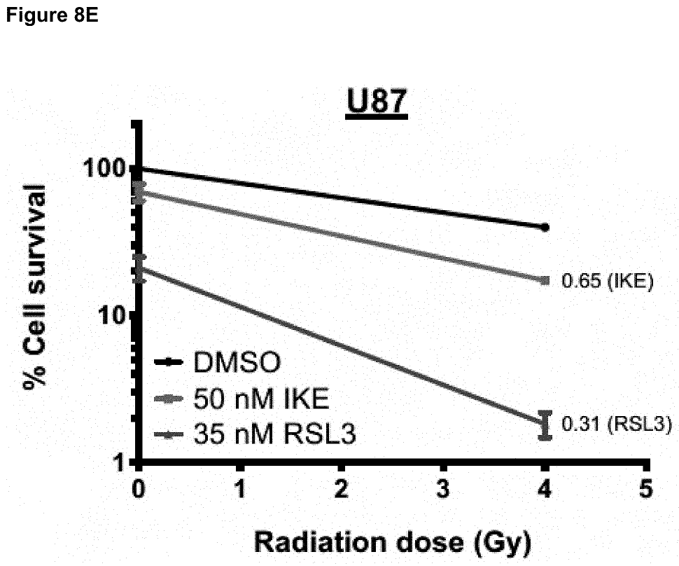

To determine first whether small molecule inducers of ferroptosis could synergize with radiation to promote cancer cell killing, ferroptosis-sensitive HT-1080 fibrosarcoma cells were treated with different doses of Cs-137 gamma radiation and either imidazole ketone erastin (IKE) or Ras Synthetic Lethal 3 (RSL3), which are small-molecule inducers of ferroptosis. We tested their ability to prevent clonogenic growth, along with DMSO-treated controls. The colony-forming ability of cells was measured, and the dose-responses to radiation of DMSO-treated, IKE-treated, and RSL3-treated groups were compared (FIG. 1A). Both IKE and RSL3 significantly enhanced the effects of radiation in decreasing clonogenic survival. Given that radiation also induces apoptosis, necroptosis, and autophagy in different contexts, it was also tested whether inducers of alternative cell death pathways could synergize with radiation under similar conditions. It's found that the apoptosis inducers staurosporine and doxorubicin, the autophagy inducer rapamycin, and induction of necroptosis using a combination of TNFα, Z-VAD-FMK and birinapant (Liu et al., 2018) were capable of only slightly enhancing radiation-induced cell death (FIG. 8A), to a lesser degree than the enhancement observed using IKE and RSL3.

The coefficient of drug interaction (CDI), used to compute interaction between two drugs, was used to quantify synergy between cell death inducers and radiation according to the formula CDI=AB/(A×B), where AB is the surviving fraction of the combination treatment, and A and B are the surviving fractions of the individual treatments. CDI <1 indicates synergy, CDI=1 indicates additivity, and CDI >1 indicates antagonism (Pikman et al., 2017) (Table 1). The results indicate that ferroptosis inducers synergize with radiation to a greater degree than other compounds in HT-1080 cells, and suggest that, although a variety of mechanisms may participate in radiation-induced cell death in this model system, ferroptosis is the most pronounced.

It was then tested whether cell death enhancement of radiation with ferroptosis inducers occurred across diverse tumor cell types. Using the same assay, we evaluated several cancer cell lines for synergistic cell killing with radiation and either IKE or RSL3 (FIGS. 8B-8E). In addition to the initial ferroptosis-sensitive HT-1080 sarcoma cell line, glioma and lung cancer cells were evaluated, due to the clinical relevance of radiation therapy for the treatment of these tumor types. Enhanced cell killing was observed in all cell lines, SK-LMS-1 (uterine sarcoma), U87 (primary glioblastoma), and A549 and PC9 (lung carcinomas) when combining radiation with a ferroptosis inducer. The CDI values for each cell line were recorded at various doses of radiation and ferroptosis inducers, and the maximal CDI for each cell line was compared (FIG. 1B and Table 2). The interactions between radiation and both ferroptosis inducers were synergistic for all the cell lines, ranging from CDI=0.70 for IKE with radiation in PC9 cells to CDI=0.09 for RSL3 with radiation in HT-1080 cells. Taken together, the results suggest that the cancer cell lines derived from radiation-sensitive tumors are synergistically killed by IKE and RSL3 and irradiation, and the enhanced radiation-induced cell killing with ferroptotic inducers may be applicable to a variety of tumor types.

TABLE 1
Coefficient of Drug Interaction (CDI) values of cell death inducers in
combination with IR in HT-1080 cells.

| Inducer | CDI with 2 Gy IR | CDI with 4 Gy IR |
|---|---|---|
| 300 nM IKE | 0.35 | 0.17 |
| 20 nM RSL3 | 0.27 | 0.09 |
| 1 nM staurosporine | 1.1 | 0.83 |
| 2 nM doxorubicin | 1.07 | 0.9 |
| 300 nM rapamycin | 0.89 | 0.62 |
| 100 ng/mL TNF + 20 μM | 0.75 | 0.65 |
| Z-VAD-FMK + 300 nM | | |
| birinapant | | |

CDI = AB/(A × B), where AB is the surviving fraction of the combination treatment, and A and B are the surviving fractions of the individual treatments. CDI < 1 indicates synergy, CDI = 1 indicates additivity, and CDI > 1 indicates antagonism.

TABLE 2
Highest observed coefficient of Drug Interaction (CDI) values of
ferroptosis inducers in combination with IR in HT-1080, SK-LMS-1,
U87, A549, and PC9 cells.

| Cell line | IKE + IR | RSL3 + IR |
|---|---|---|
| HT-1080 | 0.17 | 0.09 |
| SK-LMS-1 | 0.48 | 0.31 |
| U87 | 0.65 | 0.31 |
| A549 | 0.30 | 0.31 |
| PC9 | 0.70 | 0.64 |

CDI = AB/(A × B), where AB is the surviving fraction of the combination treatment, and A and B are the surviving fractions of the individual treatments. CDI < 1 indicates synergy, CDI = 1 indicates additivity, and CDI > 1 indicates antagonism.

### Example 3

### Radiation-Induced Cancer Cell Death is Suppressed by Ferroptosis Inhibitors

It has been reported that radiation causes lipid peroxidation in cells (Walden et al., 1988), in addition to its widely known ability to induce DNA damage. Thus, we hypothesized that cell death caused by radiation alone may partially be due to ferroptosis, particularly in contexts in which DNA damage does not induce apoptosis. To test this, we measured the effect of ferroptosis inhibitors ferrostatin-1 and deferoxamine, as well as the apoptosis inhibitor Z-VAD-FMK, on the colony-forming ability of HT-1080 cells treated with 2 or 4 Gy radiation alone. In this experiment, the lipophilic radical-trapping agent and ferroptosis inhibitor ferrostatin-1 significantly rescued colony formation, whereas the apoptosis inhibitor Z-VAD-FMK did not (FIG. 1C). Deferoxamine (DFO), a ferroptosis inhibitor and iron chelator, prevented cell proliferation and colony formation independent of radiation treatment, likely due to the requirement for iron for cell proliferation. Cells were then seeded more densely, and measured short term cell viability with an ATP-based luciferase assay to bypass this effect of DFO; cells treated with 4 Gy radiation for 24 h were rescued from death by co-treatment with either DFO or ferrostatin-1, but not by co-treatment with Z-VAD-FMK or with the necroptosis inhibitor necrostatin-1S (FIG. 1D). The autophagy inhibitor 3-methyladenine also rescued cells in this format, suggesting that autophagy may also contribute to radiation-induced cell death in this model. Given that several autophagy-related genes are positive regulators of ferroptosis, one speculative explanation is that inhibiting autophagy also limits NCOA4-dependent ferritinophagy, therefore limiting intracellular redox-active iron availability and downregulating ferroptosis (Gao et al. 2016).

It was then evaluated whether the observed synergy in cell killing between radiation and ferroptosis inducers was due to enhanced ferroptosis. In this set of colony formation assays, HT-1080 cells were treated with the same doses of radiation and ferroptosis inducers, in the presence or absence of ferroptosis inhibitors ferrostatin-1 or trolox (FIGS. 1E-1H). Both of these lipophilic radical-trapping agents (which protect lipid membranes from oxidation) acted to suppress the synergy observed between either IKE or RSL3 and radiation. Consistent with the previous experiments, both inhibitors also partially rescued cell death induced by radiation alone, in the absence of ferroptosis inducers. These results suggest that ferroptosis and lipid peroxidation contribute to radiation-induced cell death in HT-1080 cells, and that this ferroptotic cell death can be enhanced by the addition of otherwise sublethal concentrations of IKE or RSL3.

### Example 4

### Genetic and Biochemical Hallmarks of Ferroptosis are Observed in Radiation-Treated Cancer Cells

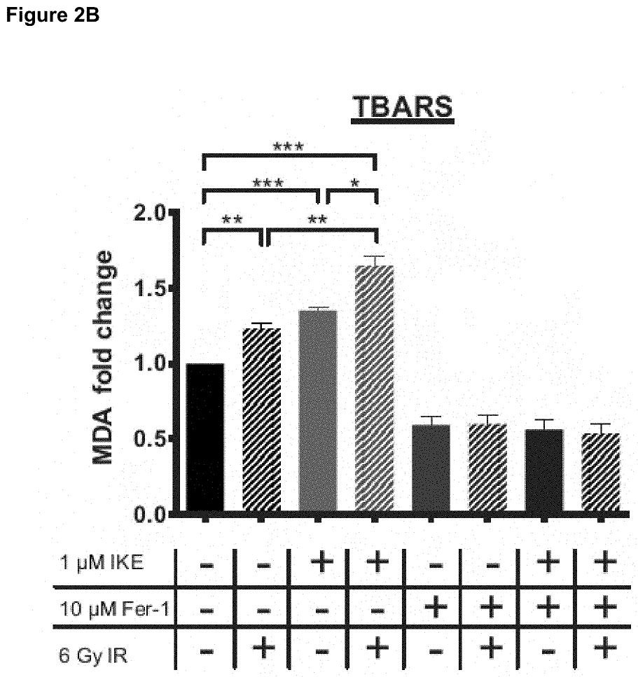

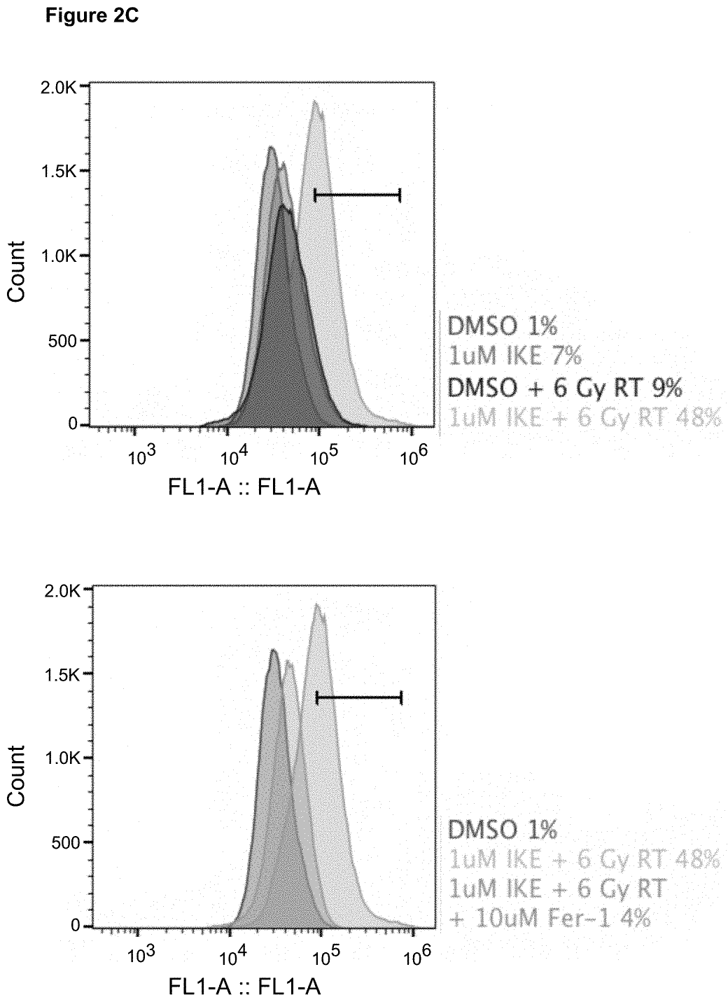

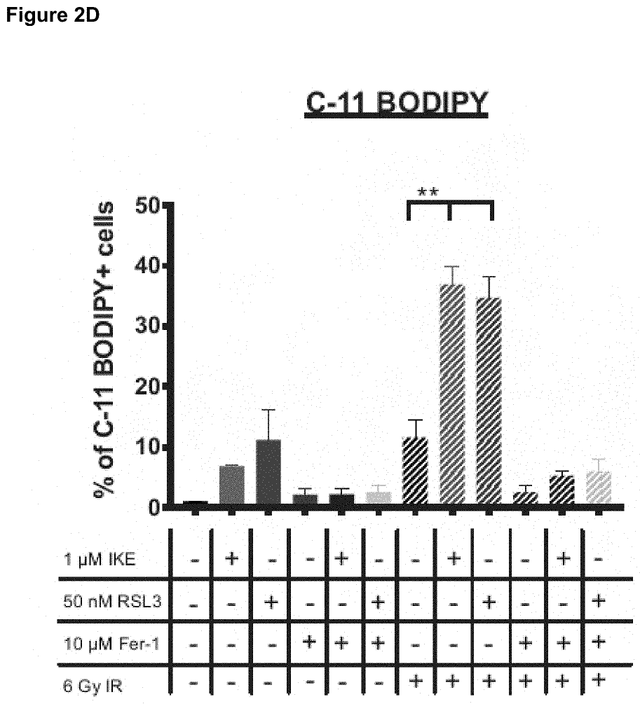

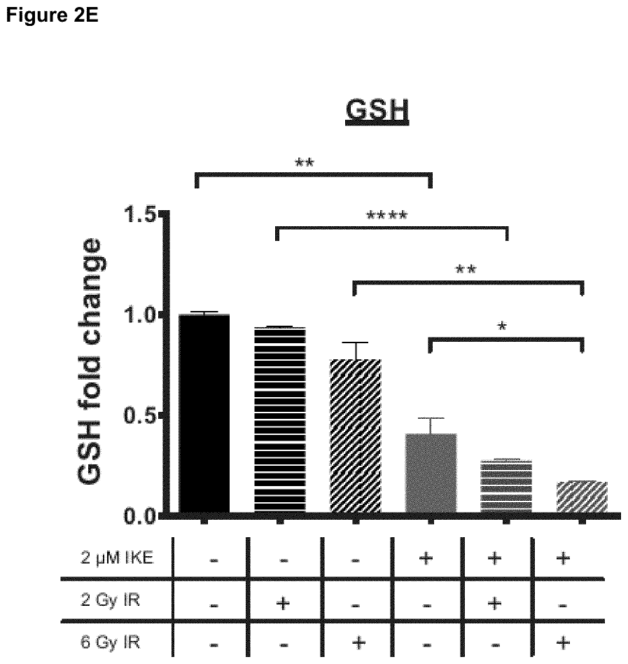

Based on the above results, to evaluate further whether ferroptosis is a mechanism for radiation-induced cell death and IKE/RSL3-amplified death in these cells, the mRNA expression level of prostaglandin-endoperoxide synthase 2 (PTGS2), a pharmacodynamic biomarker of ferroptosis (Yang et al., 2014), was measured using RT-qPCR in HT-1080 cells that were (1) radiated alone, (2) treated with 100 nM RSL3, (3) radiated and co-treated with RSL3, or (4) radiated and co-treated with 10 μM ferrostatin-1. We found that after 24 hours, PTGS2 mRNA was significantly induced in cells that were treated with 6 Gy radiation when compared to untreated cells (FIG. 2A). Treating cells with ferrostatin-1 in combination with radiation reversed this induction of PTGS2. When radiation was combined with RSL3, the upregulation in PTGS2 mRNA was even further enhanced.

Next, levels of malondialdehyde (MDA), a biomarker for lipid peroxidation and ferroptosis, were quantified using an assay that measures thiobarbituric acid reactive substances (TBARS) (Gaschler et al., 2018). In this assay, thiobarbituric acid (TBA) was added to cell lysates and heated under acidic conditions to form the MDA-TBA adduct, which was measured colorimetrically. MDA levels were found to be significantly elevated in cells treated for 24 h with 1 μM IKE, 6 Gy radiation, or a combination of the two, when compared to untreated cells (FIG. 2B). Cells treated with 10 μM ferrostatin-1, either in the presence or absence of radiation, showed significantly lower levels of MDA compared to control cells.

To confirm that radiation causes lipid peroxidation in these cells, lipid peroxidation was also measured with C-11 BODIPY (581/591), a membrane-targeted lipid sensor dye. Flow cytometry analysis of HT-1080 cells treated with radiation, ferroptosis inducers, or a combination of both for 24 h and stained with C11-BODIPY showed that the combination treatment of either 1 μM IKE or 50 nM RSL3 with 6 Gy radiation significantly increased C-11 BODIPY fluorescence when compared to either radiation or ferroptosis inducer alone (FIGS. 2C and 2D). The resulting enhancement was reversed in both cases by also co-treating the cells with ferrostatin-1.

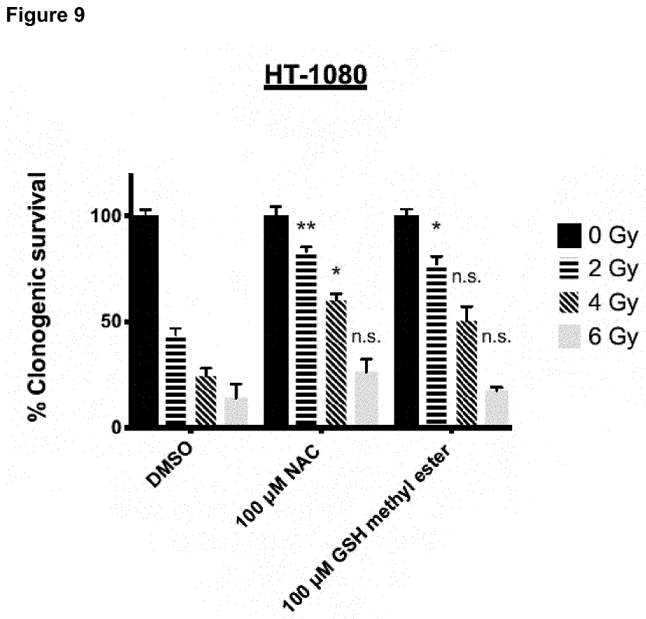

Ferroptosis inducers have been shown to alter the availability and consumption of intracellular reduced glutathione (GSH). Class I ferroptosis inducers, such as IKE, inhibit system x_{c}⁻, the cystine/glutamate antiporter on the plasma membrane that exchanges intracellular glutamate and extracellular cystine (Dixon et al., 2012; Dixon et al., 2014; Yang and Stockwell, 2016). Cystine taken up by system x_{c}⁻ is reduced to cysteine, a building block in the biosynthesis of glutathione. The glutathione-depleting effect of IKE is thought to be its main mechanism of action that drives ferroptosis. Using a fluorometric GSH probe, we observed that treatment with 2 or 6 Gy radiation for 24 hours depleted GSH in a dose-dependent manner in HT-1080 cells (FIG. 2E). In addition, levels of reduced glutathione further decreased when irradiated cells were co-treated with 2 μM IKE, suggesting that the two processes work in a cooperative fashion to deplete GSH. Indeed, the decrease in colony formation of HT-1080 cells following 2 or 4 Gy radiation was rescued by either glutathione methyl ester or N-acetylcysteine, which is a biological precursor to glutathione (FIG. 9). This finding provides a potential mechanism by which radiation and IKE act together to cause increased cell death.

### Example 5

### IKE and RSL3 do not Enhance Radiation-Induced DNA Damage Signaling or Caspase-3 Cleavage

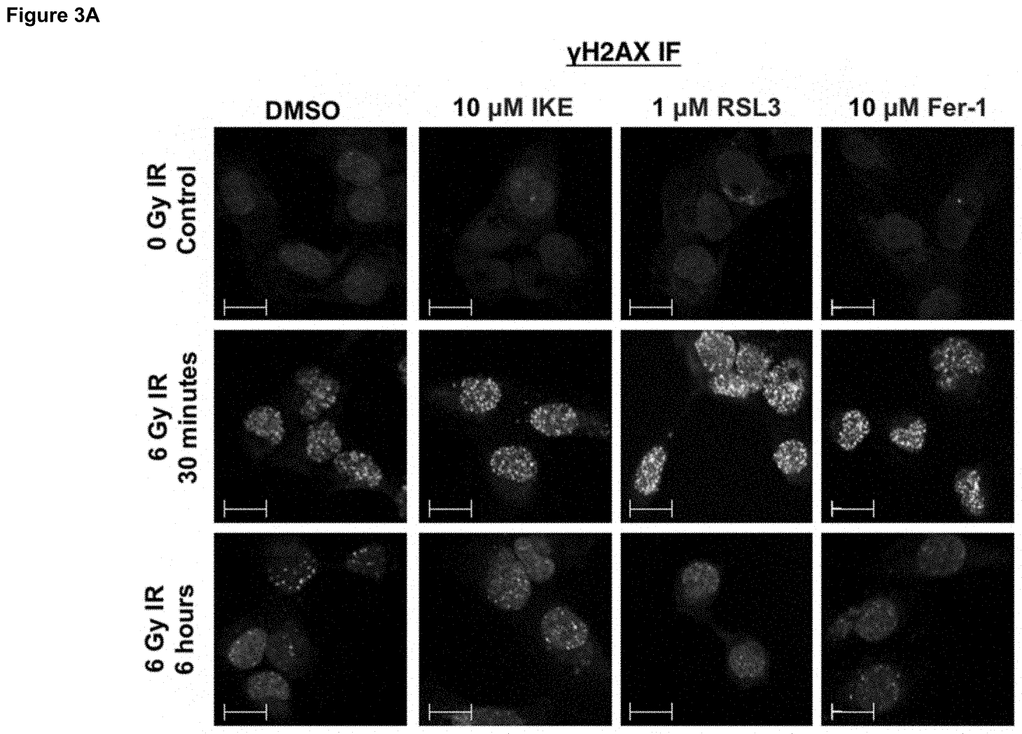

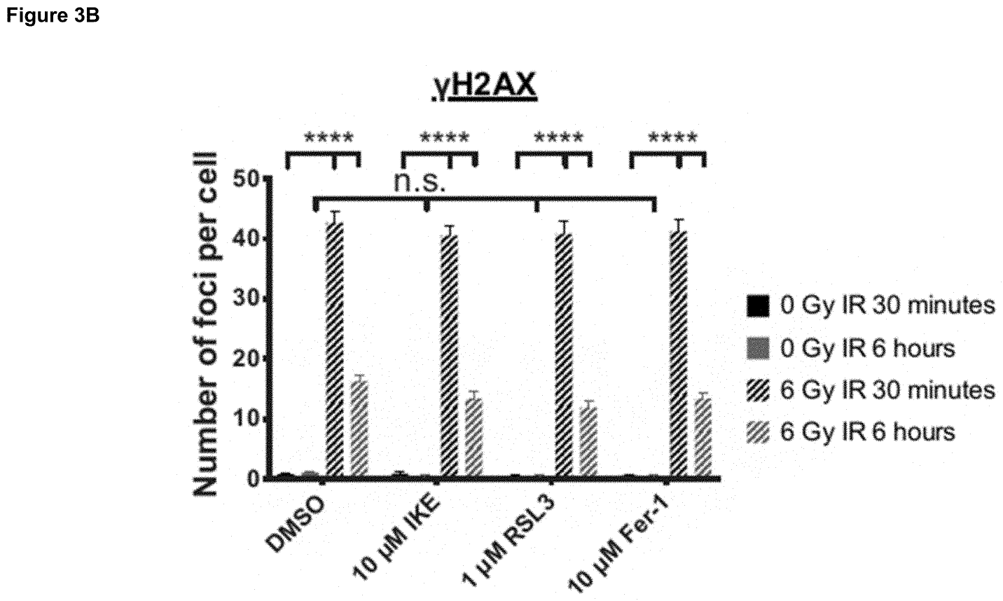

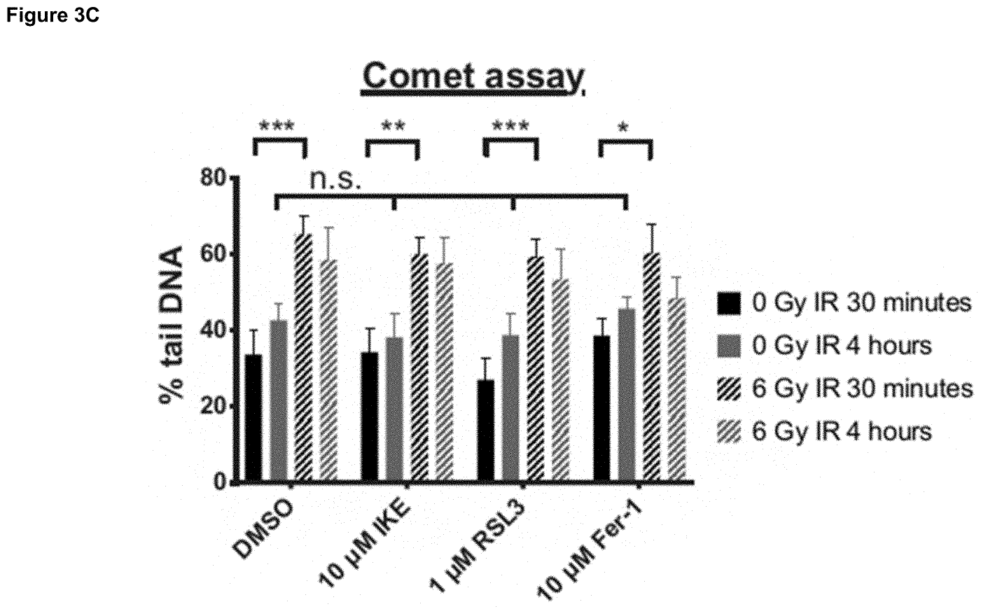

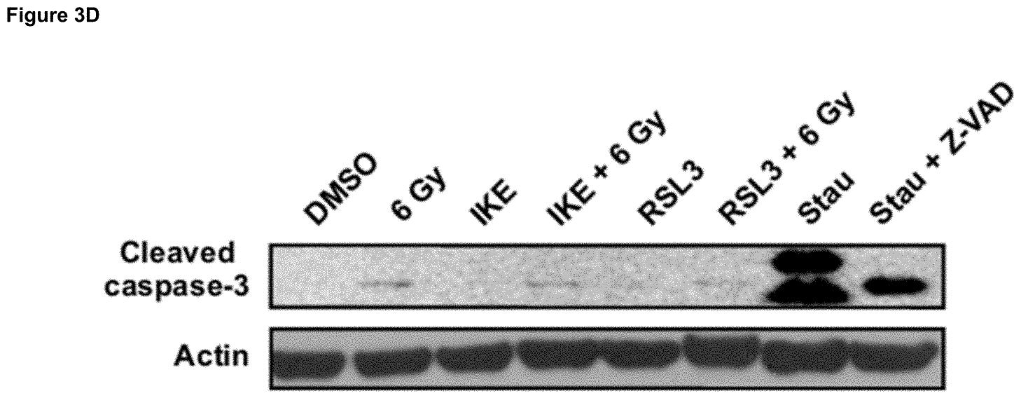

Mechanisms of the cellular lethality from radiation are thought to be mainly derived from the downstream caspase-dependent apoptosis induced by DNA damage, including complex double-strand DNA breaks (Lomax et al., 2013; Morgan and Lawrence, 2015). Therefore, it was desired to determine if ferroptosis inducers and radiation induce DNA damage or affect DNA repair. The extent of these effects of radiation was evaluated by measuring DNA damage and caspase activation in HT-1080 cells co-treated with radiation and ferroptosis inducers. Immunofluorescence staining of γH2AX, a marker for double-strand DNA (dsDNA) damage and repair, was performed in cells treated with 10 μM IKE, 1 μM RSL3, or 10 μM ferrostatin-1 along with DMSO control (FIGS. 3A, 3B). The cells were either treated with radiation (6 Gy) or not irradiated (0 Gy) as a control. After 30 minutes, numerous γH2AX foci were present in irradiated cells, but absent in control cells, suggesting that, as expected, radiation at this dose caused significant DNA damage.

However, pharmacological modulators of ferroptosis did not affect the number of observed γH2AX foci in any of the treatment groups, indicating that ferroptosis inducers alone do not cause DNA damage, and that DNA damage does not correlate with the radiation-sensitizing effects of IKE and RSL3, nor with the rescuing effect of ferrostatin-1, towards radiation-induced cell death. An experiment was then performed at 6 h post-treatment, at which point the majority of the γH2AX foci in cells treated with radiation alone disappeared, presumably due to DNA repair. Similarly, no differences were observed between the irradiated groups treated with the vehicle DMSO and those treated with IKE, RSL3, or ferrostatin-1, which suggested that DNA repair was not delayed by the co-treatment with ferroptosis inducers, nor enhanced by the co-treatment with ferrostatin-1. Treatment with IKE or RSL3 alone for 6 h did not result in γH2AX foci formation, with results similar to the 30-minute treatment. These results demonstrate that DNA damage does not correlate with the effects of ferroptosis inducers on cell viability in HT-1080 cells.

To test for other forms of DNA damage that cannot be detected by the γH2AX assay, a comet assay, which detects single strand DNA damage in addition to double strand breaks, was performed in HT-1080 cells treated under the same conditions. After 30 minutes, we observed a significant difference in percent of tail DNA between irradiated and unirradiated groups, demonstrating that DNA damage had occurred following radiation treatment. It was not detect a significant increase in DNA single strand damage in cells treated with IKE or RSL3 alone, and no significant enhancement of DNA damage when IKE or RSL3 was combined with radiation, even when a proportion of cells had started to die at the 4-hour timepoint (FIG. 3C). We also did not observe a significant protective effect of ferrostatin-1 towards radiation-induced DNA single strand damage. These results again reinforce the conclusion that although DNA damage occurs in HT-1080 cells exposed to radiation, it is not related to the synergistic cell death observed during co-treatment with ferroptosis inducers.

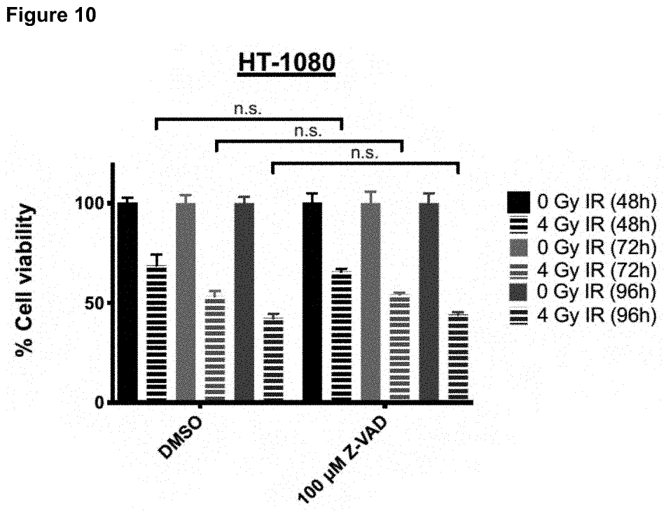

DNA damage is a potent inducer of apoptosis. Therefore, the presence of radiation-induced apoptosis was also tested by measuring levels of cleaved caspase-3 in HT-1080 cells treated with 6 Gy radiation, or with IKE or RSL3, or with a combination of radiation plus ferroptosis inducer, for 24 h (FIG. 3D). Levels of cleaved caspase-3 were minimally elevated in cells treated with radiation compared with those of non-irradiated cells, and the addition of ferroptosis inducers did not further increase the amount of cleaved caspase-3. Treatment with ferroptosis inducers alone did not induce detectable cleavage of caspase-3, as previously reported (Yagoda et al., 2007). In contrast, the pro-apoptosis inducer staurosporine, used as a positive control at 500 nM for 6 h, induced cell death along with levels of caspase-3 cleavage, shown by bands at 17 and 19-kDa. When these cells were co-treated with staurosporine and 100 μM Z-VAD-FMK, a pan caspase inhibitor, the cells were rescued from staurosporine-induced cell death. Despite this, some quantity of 17-kDa cleaved caspase-3 was still detectable in the Z-VAD-FMK-treated sample. To further check for potential radiation-induced apoptosis in this model at a later time point, we attempted to rescue the effects of radiation using 100 μM Z-VAD-FMK after 48, 72, and 96 h (FIG. 10). However, no significant rescue of cell viability was observed at any of these time points.

These findings suggest that ferroptosis driven by lipid peroxidation, not DNA damage or apoptosis, is the predominant radiosensitizing mechanism of ferroptosis inducers, and of radiation on its own, in HT-1080 cells.

### Example 6

### Untargeted Lipidomics Reveals Molecular Features of Ferroptosis in Cells Co-Treated with IKE and Radiation

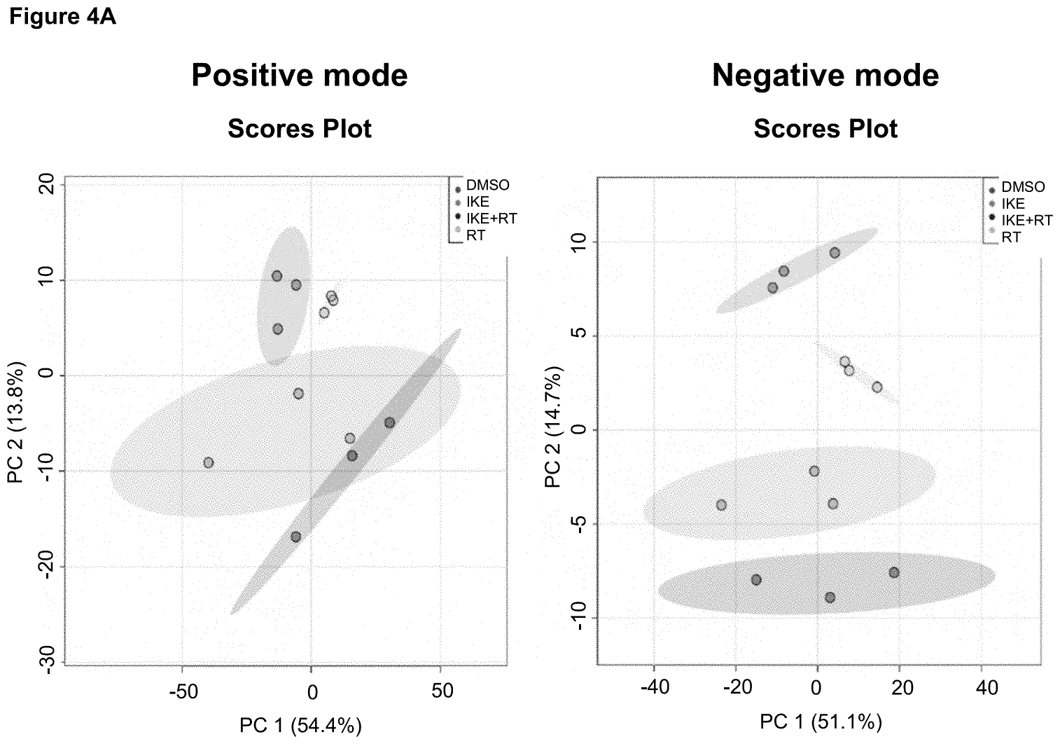

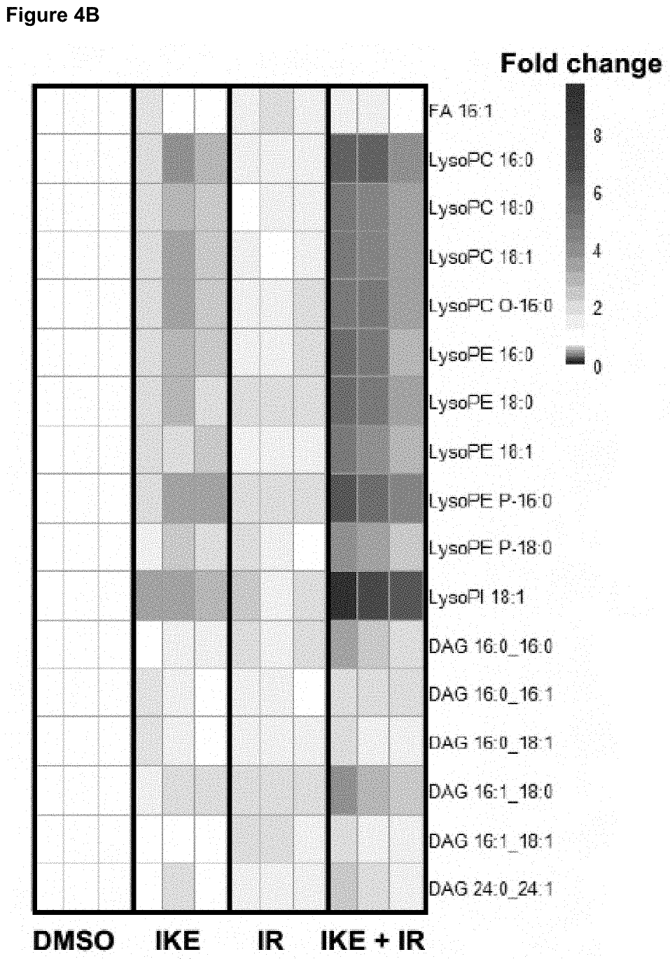

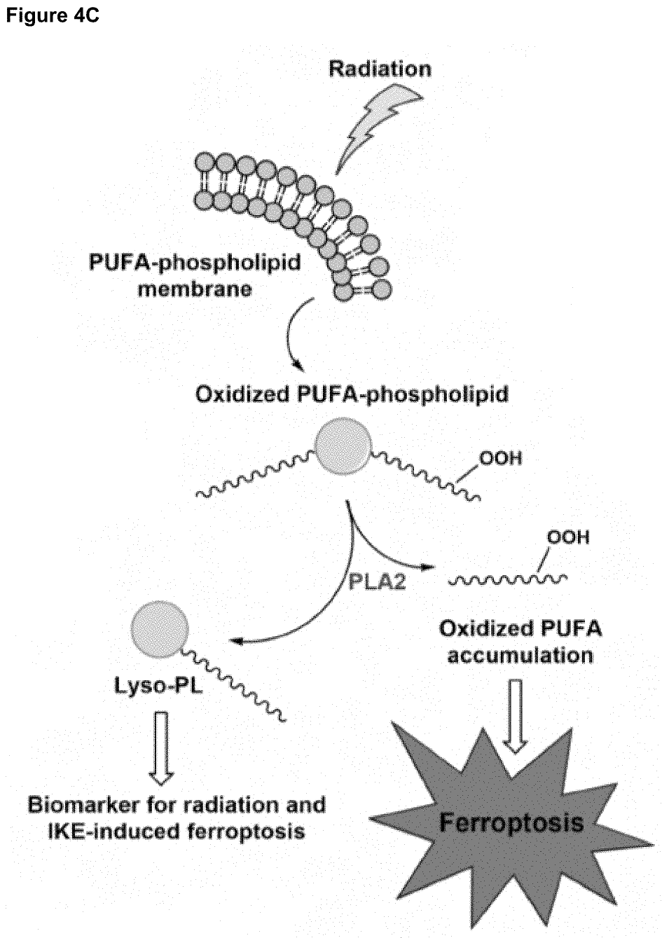

To further probe the effects of radiation on cellular lipid composition and metabolism, it was performed an ultra-performance liquid chromatography quadrupole-time-of-flight-mass-spectrometry-(UPLC-q-ToF MS−) based untargeted lipidomics analysis of HT-1080 cells treated with 0 or 6 Gy radiation for 24 h, and co-treated with either DMSO vehicle or 5 μM IKE for 12 h. Unsupervised principal component analysis of the detected lipid features in both positive and negative electrospray ionization modes showed clear clustering and separation among the groups (FIG. 4A). Using two-way ANOVA (FDR corrected p value <0.05 for both IKE-treated and IR-treated samples when compared to control samples), we found 18 lipid ions in the positive and 10 lipid ions in the negative ESI modes whose abundances changed significantly among the groups (Table 3). By integrating the annotated lipid ions in both modes, we found 17 unique lipid species, including one free fatty acid (FA 16:1), 10 lysophospholipids (LysoPLs), and six diacylglycerols (DAGs), which increased significantly in cells treated with IKE or radiation, with even larger increases when IKE and radiation were combined (FIG. 4B). Lysophospholipids, molecules generated following PUFA-containing phospholipid peroxidation by enzymatic cleavage of the oxidized PUFA tail, have been implicated in oxidative stress and accumulate during treatment with ferroptosis inducers (Colles and Chisolm, 2000; Yang et al., 2016; Zhang et al., 2019). Among these, lysophosphatidylinositol (LysoPI) 18:1 (interaction p value=0.01) and lysophosphatidylethanolamine (LysoPE) 18:1 (p=0.03) in particular had significantly interacting synergistic effects between IKE and radiation. Furthermore, the significantly elevated levels of diacylglycerols may have resulted from hydrolysis of triacylglycerols, which are enriched in ferroptosis-sensitive cell states of clear-cell carcinoma and have been shown to be accumulated by IKE in cell culture models of diffuse large B-cell lymphoma (Zhang et al., 2019; Zou et al., 2019). Of these, DAG 16:0/16:1 also displayed significant interaction between IKE and radiation (p<0.05).

These results suggest that radiation-driven lipid peroxidation produces a downstream lipid signature similar to that produced by IKE alone, and consistent with the previous studies of cell lipidome changes during ferroptosis. In combination therapy, the oxidation of PUFA-phospholipids by radiation enhances the same effect driven by IKE, potentiating ferroptosis and producing lysophospholipids as a by-product that are as a ferroptosis biomarker (FIG. 4C). Next a precision charged particle microbeam was utilized to elucidate the consequence of lipid peroxidation with irradiation of sub-cellular compartments.

TABLE 3
List of the annotated lipids that significantly changed among the groups
(Two-way ANOVA; FDR-corrected p-value < 0.05), including ionization mode as
detected adducts (positive/negative), retention time (RT), m/z values, mass error
(Δppm), molecular formula, and major product ions.

| | | | | Mass | | |
|---|---|---|---|---|---|---|
| | Ionization | Retention | m/z | error | Molecular | Major detected |
| Lipids | mode | time | observed | (ΔPPM) | formula | product ions |
| FA 16:1 | [M − H]⁻ | 1.99 | 253.2162 | 4 | C₁₆H₃₀O₂ | 235.2 |
| LysoPE | [M − H]⁻ | 1.42 | 452.2771 | 2 | C₂₁H₄₄NO₇P | 255.2 |
| 16:0 | | | | | | |
| LysoPE | [M − H]⁻ | 2.06 | 480.3084 | 2 | C₂₃H₄₈NO₇P | 283.2 |
| 18:0 | | | | | | |
| LysoPE | [M − H]⁻ | 1.49 | 478.2925 | 2 | C₂₃H₄₆NO₇P | 281.2 |
| 18:1 | | | | | | |
| LysoPE | [M − H]⁻ | 1.62 | 436.282 | 3 | C₂₁H₄₄NO₆P | 418.2 |
| P-16:0 | | | | | | |
| LysoPE | [M − H]⁻ | 2.39 | 464.3134 | 2 | C₂₃H₄₆NO₆P | 446.2 |
| P-18:0 | | | | | | |
| LysoPI | [M − H]⁻ | 1.02 | 597.3045 | 0 | C₂₇H₅₁O12P | 281.2/241.2 |
| 18:1 | | | | | | |
| LysoPC | [M + CH₃COO]⁻ | 1.34 | 554.3452 | 2 | C₂₄H₅₀NO₇P | 480.3/255.2 |
| 16:0 | | | | | | |
| LysoPC | [M + CH₃COO]⁻ | 1.93 | 582.3767 | 1 | C₂₆H₅₄NO₇P | 508.3/283.2 |
| 18:0 | | | | | | |
| LysoPC | [M + CH₃COO]⁻ | 1.42 | 580.3615 | 0 | C₂₆H₅₂NO₇P | 506.3/281.2 |
| 18:1 | | | | | | |
| DAG | [M + H − H₂O]⁻ | 13.69 | 551.504 | 0 | C₃₅H₆₈O₅ | 313.3 |
| 16:0_16:0 | | | | | | |
| DAG | [M + H − H₂O]⁻ | 12.95 | 549.4883 | 0 | C₃₅H₆₆O₅ | 313.3/311.3 |
| 16:0_16:1 | | | | | | |
| DAG | [M + H − H₂O]⁻ | 13.74 | 577.5191 | 0 | C₃₇H₇₀O₅ | 313.3/339.3 |
| 16:0_18:1 | | | | | | |
| DAG | [M + H − H₂O]⁻ | 14.30 | 577.5167 | 4 | C₃₇H₇₀O₅ | 341.3 |
| 16:1_8.0 | | | | | | |
| LysoPC16:0 | [M + H]⁺ | 1.36 | 496.3407 | 1 | C₂₄H₅₀NO₇P | 184.07/(Acyl chain |
| | | | | | | was confirmed in |
| | | | | | | negative mode; |
| | | | | | | 255.2) |
| LysoPC | [M + H]⁺ | 1.93 | 524.3717 | 1 | C₂₆H₅₄NO₇P | 184.07/(Acyl chain |
| 18:0 | | | | | | was confirmed in |
| | | | | | | negative mode; |
| | | | | | | 283.2) |
| LysoPC | [M + H]⁺ | 1.42 | 522.3506 | 2 | C₂₆H₅₂NO₇P | 184.07/(Acyl chain |
| 18:1 | | | | | | was confirmed in |
| | | | | | | negative mode; |
| | | | | | | 253.2) |
| LysoPC | [M + H]⁺ | 1.59 | 482.3611 | 1 | C₂₄H₅₂NO6P | 184.07/104.1 |
| O-16:0 | | | | | | |
| LysoPE | [M + H]⁺ | 2.05 | 482.3247 | 1 | C₂₃H₄₈NO₇P | Neutral loss of |
| 18:0 | | | | | | 141.2/Acyl chain |
| | | | | | | was confirmed in |
| | | | | | | negative mode; |
| | | | | | | 283.2) |
| LysoPE | [M + H]⁺ | 1.49 | 480.3104 | 4 | C₂₃H₄₈NO₇P | Neutral toss of |
| 18:1 | | | | | | 141.2/Acyl chain |
| | | | | | | was confirmed in |
| | | | | | | negative mode; |
| | | | | | | 281.2) |
| LysoPE | [M + H]⁺ | 1.62 | 438.2983 | 0 | C₂₁H₄₄NO₆P | 242.2 |
| P-16:0 | | | | | | |
| LysoPE | [M + H]⁺ | 2.40 | 466.3296 | 0 | C₂₃H₄₈NO₆P | 325.2 |
| P-18:0 | | | | | | |
| DAG | [M + Na]⁺ | 13.69 | 591.496 | 0 | C₃₅H₆₈O₅ | 313.3 |
| 16:0_16:0 | | | | | | |
| DAG | [M + NH₄]⁺ | 13.69 | 586.5407 | 0 | C₃₅H₆₈O₅ | 313.3 |
| 16:0_16:0 | | | | | | |
| DAG | [M + NH₄]⁺ | 12.95 | 584.5253 | 0 | C₃₆H₆₆O₅ | 313.3/311.3 |
| 16:0_16:1 | | | | | | |
| DAG | [M + NH₄]⁺ | 13.73 | 612.5563 | 0 | C₃₇H₇₀O₅ | 313.3/339.3 |
| 16:0_18:1 | | | | | | |
| DAG | [M + NH₄]⁺ | 13.34 | 610.5395 | 1 | C₃₇H₆₈O₅ | 311.3/339.3 |
| 16:1/18:1 | | | | | | |
| DAG | [M + NH₄]⁺ | 17.40 | 808.7745 | 0 | C₅₁H₉₈O₅ | 425.4 |
| 24:0_24:1 | | | | | | |

### Example 7

### Targeted Cytoplasmic, but not Nuclear, Microbeam Radiation Selectively Synergizes with IKE and RSL3 to Enhance Clonogenic Cell Death

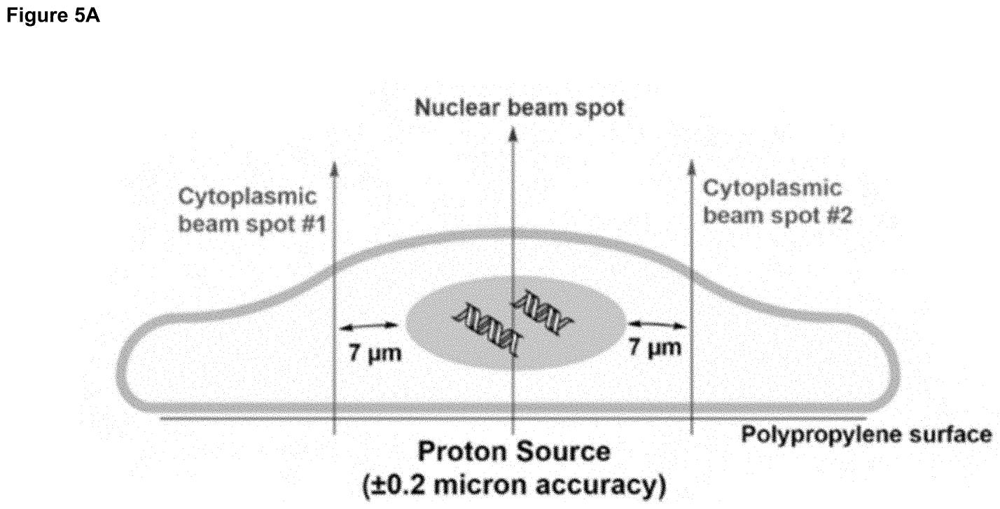

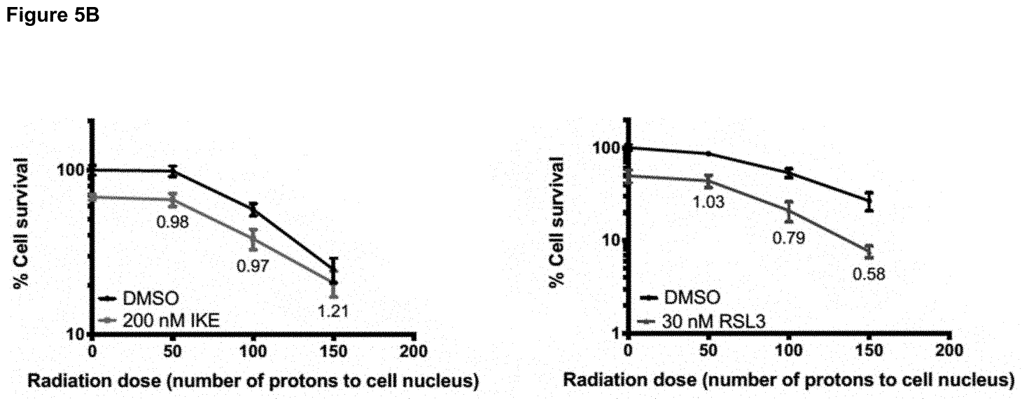

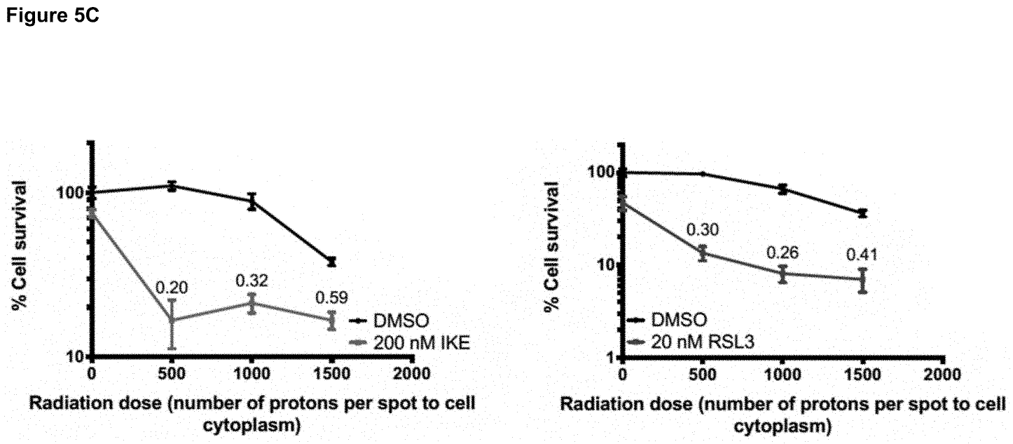

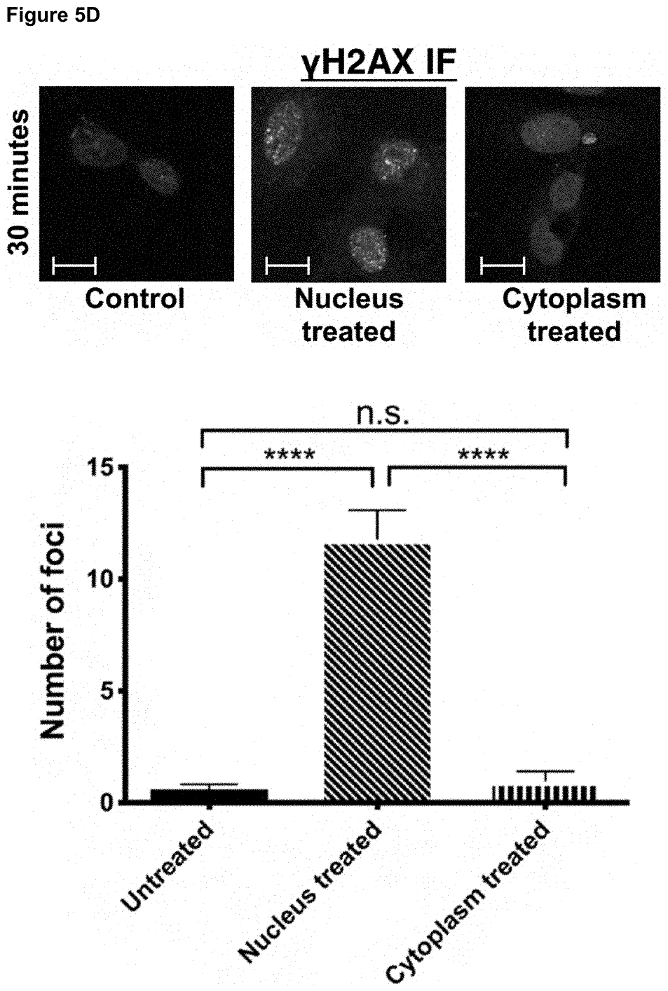

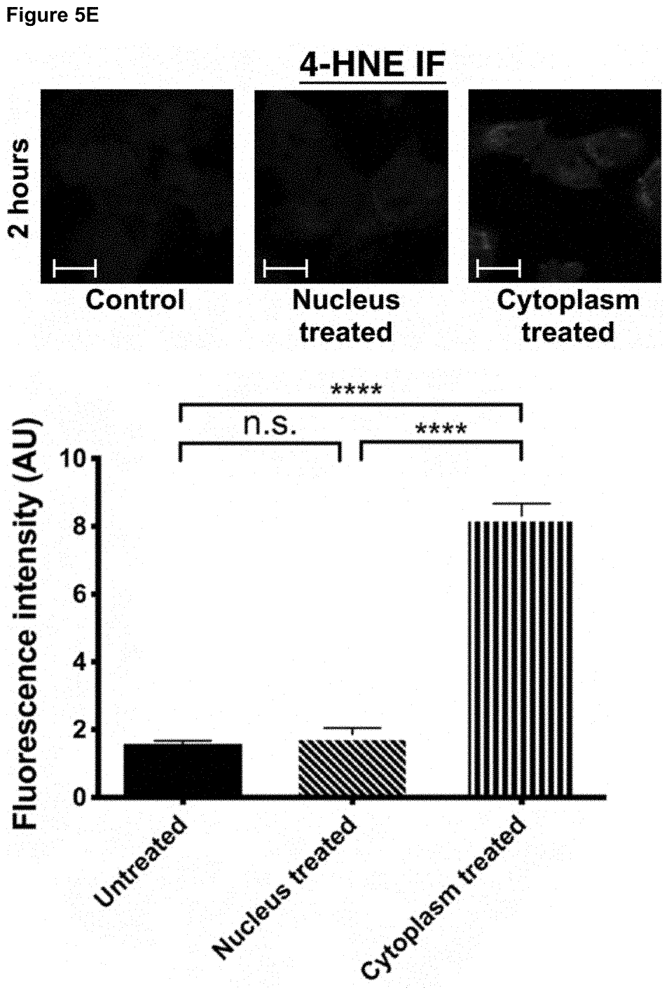

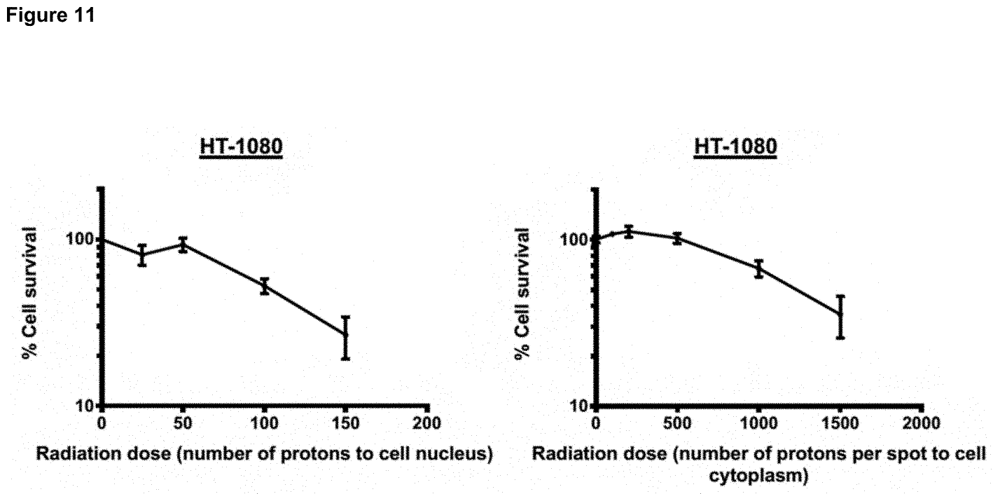

To further probe the mechanism by which radiation synergizes with IKE and RSL3 to cause cell death, a 5-MeV proton microbeam was used to deliver targeted radiation to either the nucleus or the cytoplasm of HT-1080 cells (Randers-Pehrson et al., 2009). The microbeam consists of a single beam of proton radiation with a spot size of 4 micrometers that allows radiation damage to be precisely deposited at specific locations in a cell. This translates to delivery of a precise number of protons to either the cell nucleus or to the cytoplasm outside of the nucleus (Hei et al., 1997; Wu et al., 1999). The targetable nature of the microbeam allows us to distinguish the cytoplasmic effects of radiation from its nuclear effects, and test if the former is the predominant component that drives radiation-induced ferroptosis. To target the microbeam, cells were labeled with Hoechst stain and imaged. Nuclear radiation was delivered to the center of gravity of the cell nucleus, whereas cytoplasmic radiation was delivered to two sites 7 microns away from the nuclear edge along the nuclear long axis (FIG. 5A). Using this method, we first established dose responses of these cells to nuclear and cytoplasmic radiation. The ED₅₀ for clonogenic cell death was observed to be around 100 protons for nuclear radiation, and between 1,000 and 1,500 protons per site for cytoplasmic radiation (FIG. 11). Compared to conventional photon radiation, these doses approximately correspond to 1 Gy to the nucleus and between 1-5 Gy to the cytoplasm, a therapeutically relevant dose range that is consistent with our previous experiments. Similar to previous reports, these results suggest that nuclear proton radiation was more lethal to cells than cytoplasmic proton radiation, presumably through direct radiation-induced damage to DNA and genome integrity (Zhou et al., 2009). This supports the view that the genotoxic effects of radiation are attributed mainly to direct damage to the nucleus.

However, when microbeam radiation was combined with inducers of ferroptosis, we observed that nuclear radiation had no synergy with IKE and RSL3, whereas cytoplasmic radiation synergized strongly with both compounds (FIGS. 5B and 5C). Notably, although no significant cell death was observed with 500 or 1,000 protons alone delivered per site to the cytoplasm, there was a large decrease in cell survival when irradiated cells were concurrently treated with sub-lethal doses of either of the two ferroptosis inducers, leading to synergistic CDI values between 0.2 and 0.4. By comparison, no such effect was observed when the cells were treated with nuclear irradiation. Taken together, these results suggest that ferroptosis inducers sensitize cells to the effects of radiation primarily in the cytoplasm.

To further highlight the differences between nuclear and cytoplasmic microbeam radiation, and to examine if they represent two distinct forms of radiation-induced cell death, levels of DNA damage and lipid peroxidation were measured in cells treated under the two conditions. To measure DNA damage, cells were treated either with 100 protons to the nucleus or 2,000 protons to each site in the cytoplasm. γH2AX immunofluorescence staining for dsDNA breaks was performed 30 minutes post-irradiation (FIG. 5D). γH2AX foci were indeed present in cells treated with nuclear radiation, but absent from cells treated with cytoplasmic radiation. To examine the microbeam's effects on lipid peroxidation, we performed immunofluorescence staining of 4-hydroxynonenal (4-HNE), a marker of lipid peroxidation, in cells with the same treatment conditions at 2 hours post-irradiation (FIG. 5E). The 4-HNE signal was significantly increased in samples treated with cytoplasmic radiation relative to untreated cells, but not in those treated with nuclear radiation.

Taken together, these findings indicate that, although damage to the nucleus remains an important mechanism of radiation therapy in some contexts, inducers of ferroptosis serve to activate a distinct cell death mechanism based in the cytoplasm, which may become relevant in cancer cells that have acquired resistance to the traditional cell death and DNA damage pathways. Although it is not possible to differentially deliver cytoplasmic vs nuclear radiation in clinical contexts, the microbeam is nevertheless a useful tool to separate the effects of radiation-induced lipid peroxidation and DNA damage and to examine how ferroptosis synergizes with the former but not the latter in different cells and tumors. Further mechanistic studies may reveal the targets contained within the cytoplasm required for this synergy, which could in principle be pharmacologically modulated for downstream clinical applications.

### Example 8

### IKE and Sorafenib Enhance Effects of Radiation to Inhibit Tumor Growth in a Xenograft Mouse Model of Sarcoma

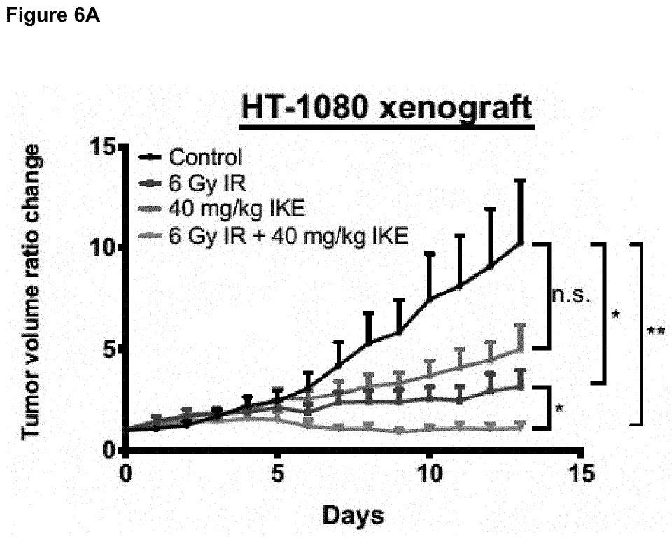

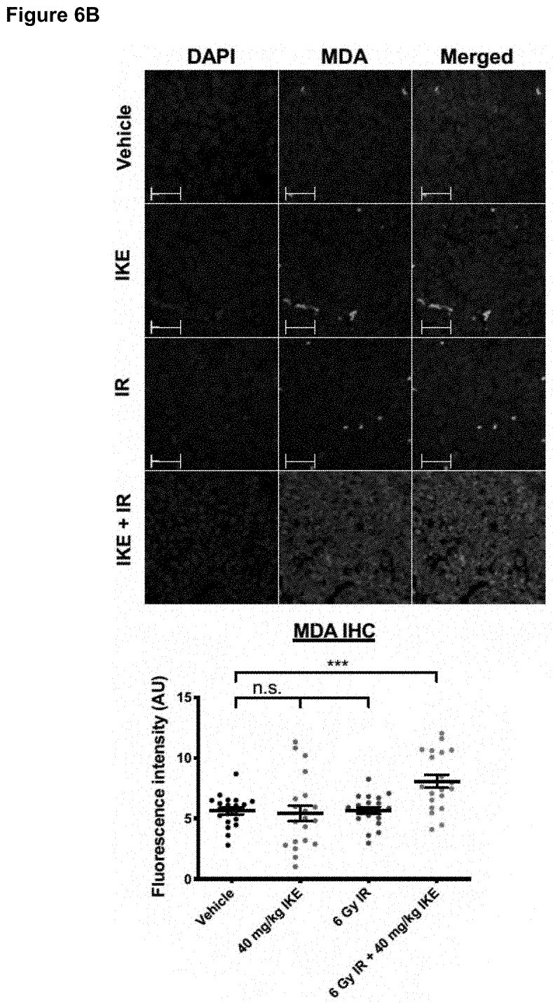

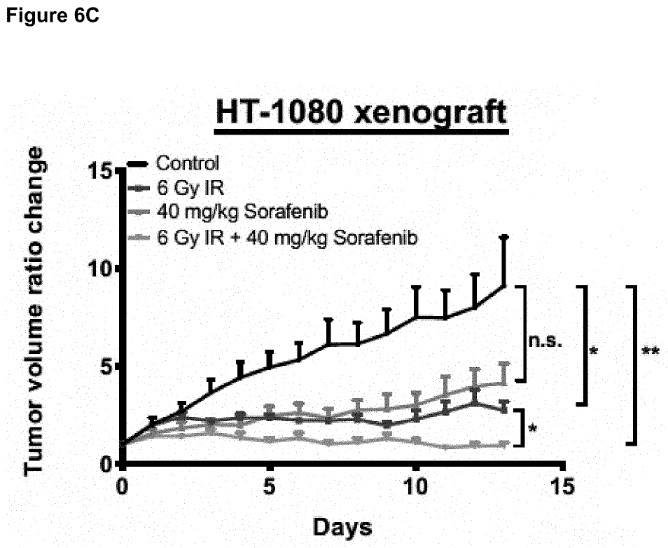

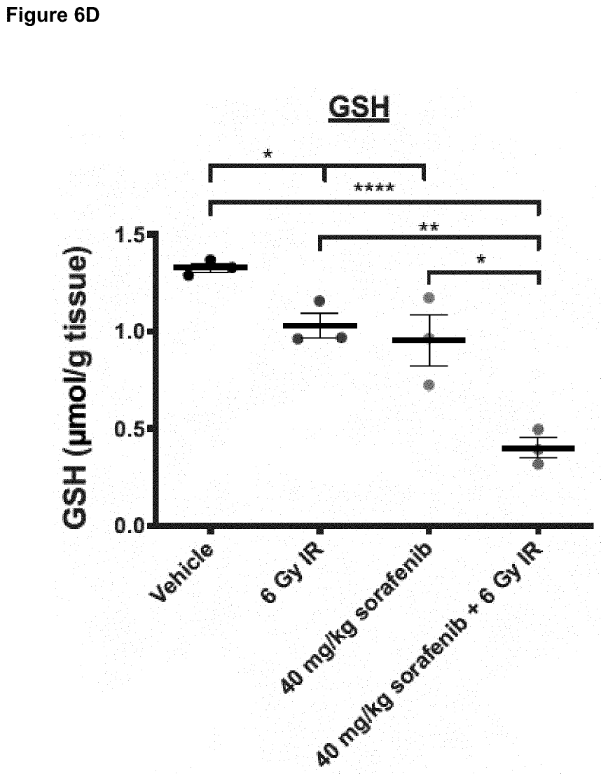

The efficacy of the combined treatment regimen of radiation and ferroptosis inducer was evaluated in an in vivo tumor model. Of the two ferroptosis inducers tested in cell culture, IKE was selected for in vivo studies due to its previously established stability and activity in xenograft mouse models of cancer (Zhang et al., 2019). Athymic nude mice were implanted with subcutaneous HT-1080 fibrosarcoma cells to form xenograft tumors. When the tumors reached an average volume of approximately 100 cubic millimeters, intraperitoneal (i.p.) injections of 40 mg/kg IKE or vehicle were delivered daily for 14 days, starting on day 0 and ending on day 13. On days two and four of IKE treatment, 0 or 6 Gy radiation was delivered to the tumor site using the Small Animal Radiation Research Platform (SARRP) system (Wu et al., 2017). After two weeks, tumor volume was compared between mice treated with vehicle, IKE alone, radiation alone, or a combination of both (FIG. 6A). Using two-sample t-tests, we observed a significant difference between the vehicle-treated control group and the groups treated with radiation alone; IKE alone was not strongly effective at this dose level in this model. We observed a significant further reduction in tumor volume between the single treatment groups when compared to the group treated with combination therapy, showing that IKE enhanced the effects of radiation in reducing tumor growth. Upon analysis of all groups with the two-way ANOVA test, we found statistical significance for treatment with IKE alone (p=0.03) and radiation alone (p=0.004). The two factors interacted positively with each other, although the interaction P value did not reach significance (p=0.34).

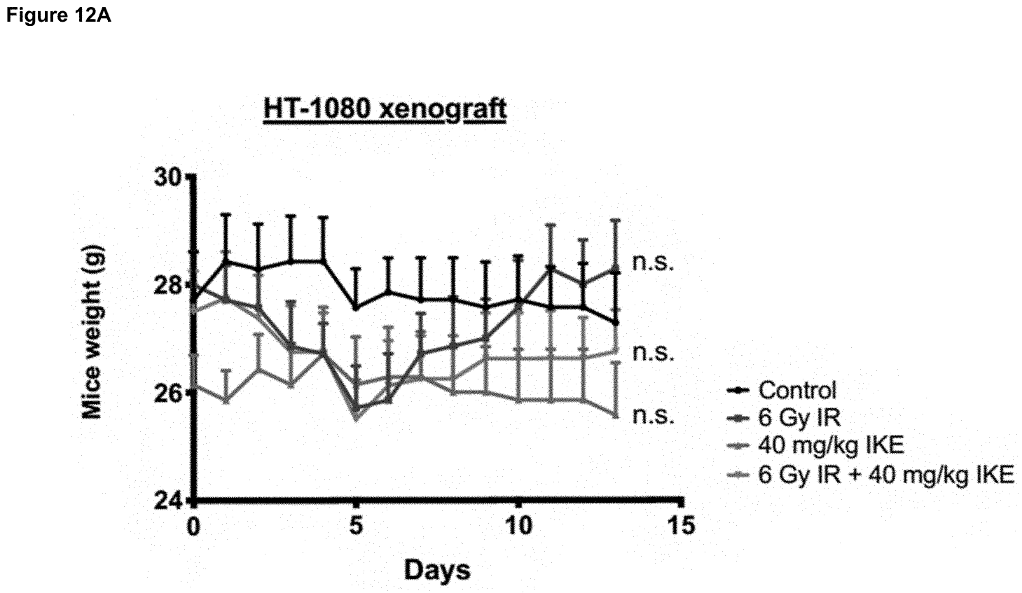

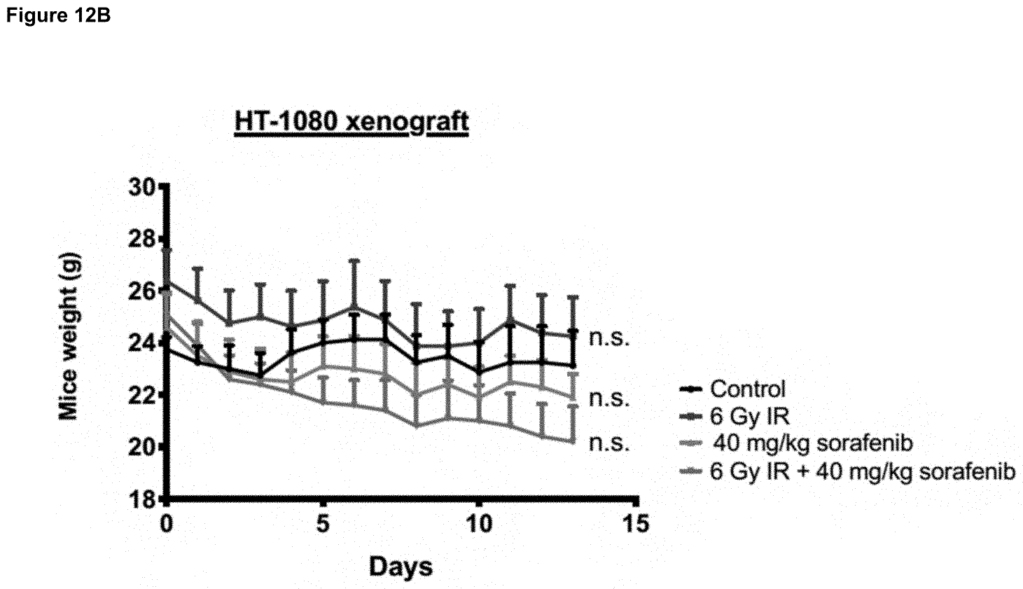

Using weight loss as a measure of mice health, no significant differences was observed between any of the groups for the duration of the experiment (FIG. 12A). This suggests that IKE and radiation and the combination were well tolerated at these dose levels for this length of exposure.

Next, malondialdehyde (MDA) levels, as a biomarker for ferroptosis, were measured in tumor tissue using immunohistochemistry on fixed and paraffin-embedded tumor samples resected at day 14 post-treatment (FIG. 6B). Significantly elevated MDA signal was observed in tumors treated with both IKE and radiation compared to that of tumors treated with vehicle, suggesting enhanced lipid peroxidation and ferroptosis in the co-treated tumors. No significant differences were observed between tumors treated with vehicle, IKE only, or radiation only. This again suggests synergistic pharmacodynamic effect of radiation and IKE in this tumor model.

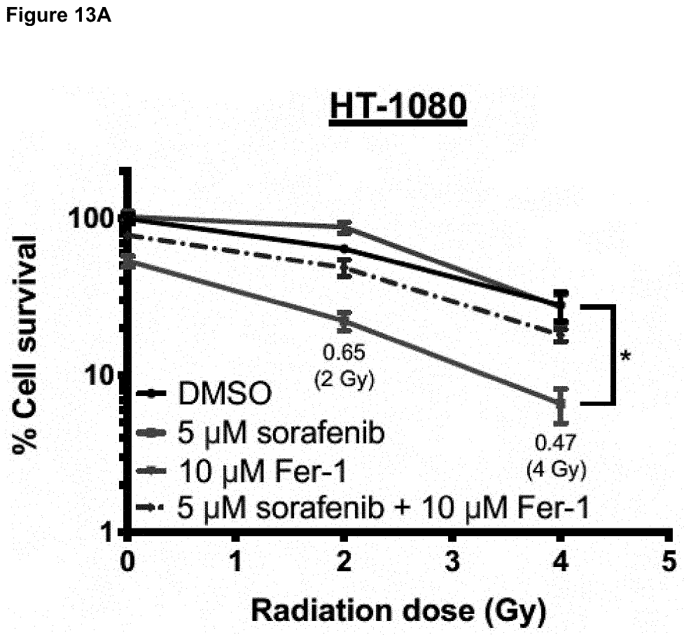

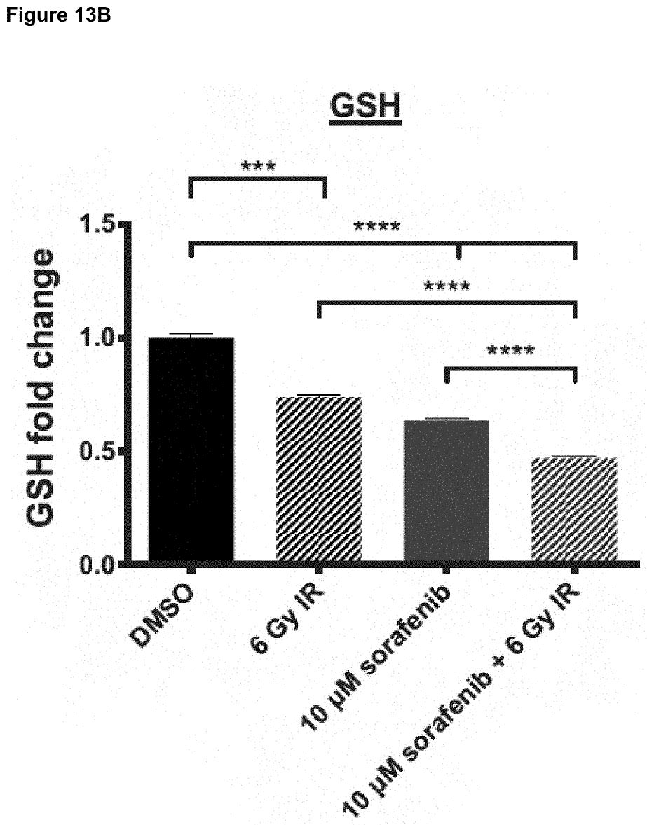

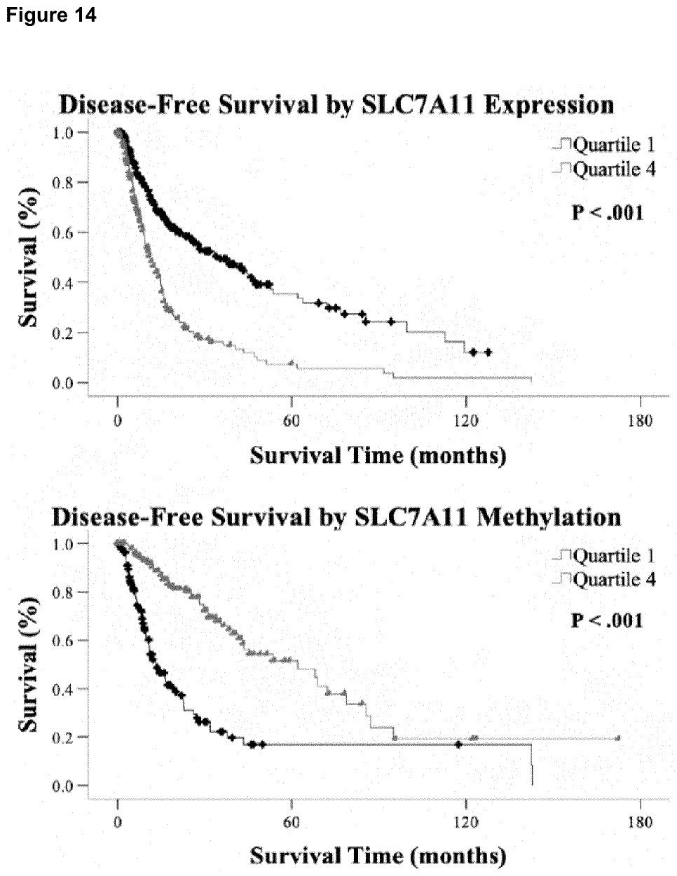

It was then tested the radiosensitizing effect of sorafenib, an FDA-approved chemotherapeutic drug, which also acts as an inhibitor of system x_{c}⁻ (Dixon et al., 2014). First, the colony-forming ability of HT-1080 cells when treated with sorafenib, radiation or a combination was compared to that of untreated cells. It's found that treatment with 5 μM sorafenib is synergistic with radiation at both 2 Gy (CDI=0.65) and 4 Gy (CDI=0.47), and that this effect is partially suppressible by co-treatment with ferrostatin-1 (FIG. 13A). To confirm that the observed synergistic effect between sorafenib and radiation in HT-1080 cells is due to system x_{c}⁻ inhibition, levels of GSH were then measured in these cells treated with DMSO or sorafenib, and co-treated with 0 or 6 Gy radiation for 24 hours. Indeed, significant depletion of GSH was observed in the dual treated sample, when compared to samples treated with DMSO, sorafenib alone, or radiation alone (FIG. 13B).

To test the radiosensitizing effects of sorafenib in vivo, athymic nude mice implanted with HT-1080 xenograft tumors, as described above, were treated with 40 mg/kg sorafenib tosylate or vehicle delivered i.p. daily for 14 days, with or without 6 Gy radiation delivered per mouse using SARRP on days 1 and 3. Using two-sample t-tests, we observed significant differences between the control, radiation-treated, and combination-treated groups (FIG. 6C). No difference was observed between the control group and the group treated with sorafenib alone. Two-way ANOVA revealed statistical significance for treatment with sorafenib alone (p=0.03) and radiation alone (p=0.006). Similar to the experiment with IKE, the two factors interacted positively with each other but did not reach significance (p=0.18). No significant differences in weight were observed between any of the groups over the course of the experiment (FIG. 12B).

To confirm that sorafenib inhibits system x_{c}⁻ in vivo, leading to a depletion of downstream intracellular reduced glutathione, GSH were measured in tumor tissue taken from three mice in each group and found significantly lower GSH in tumors treated with either radiation or sorafenib compared to those treated with vehicle. A further significant GSH depletion was observed in dual-treated tumors (FIG. 6D).

### Example 9

### Analysis of Patient Data Suggests a Role for SLC7A11 in Radioresistance of Gliomas

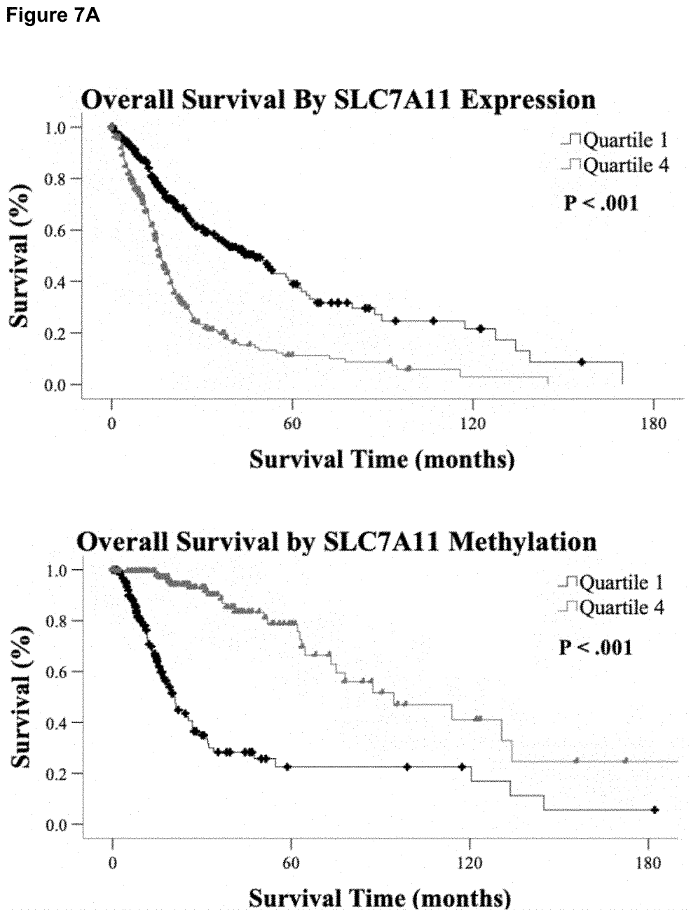

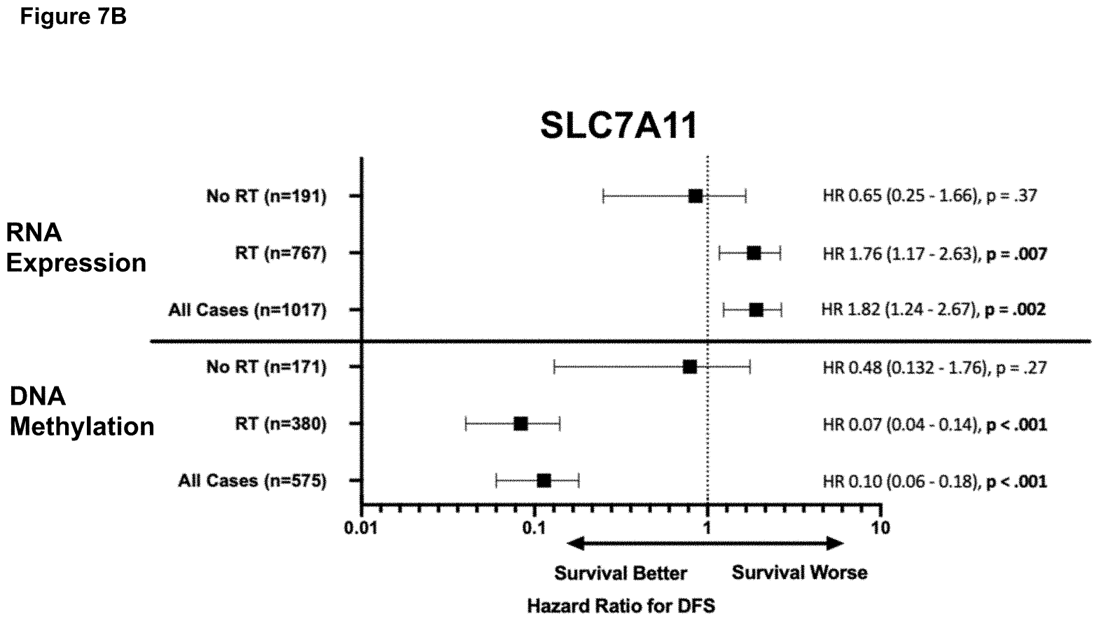

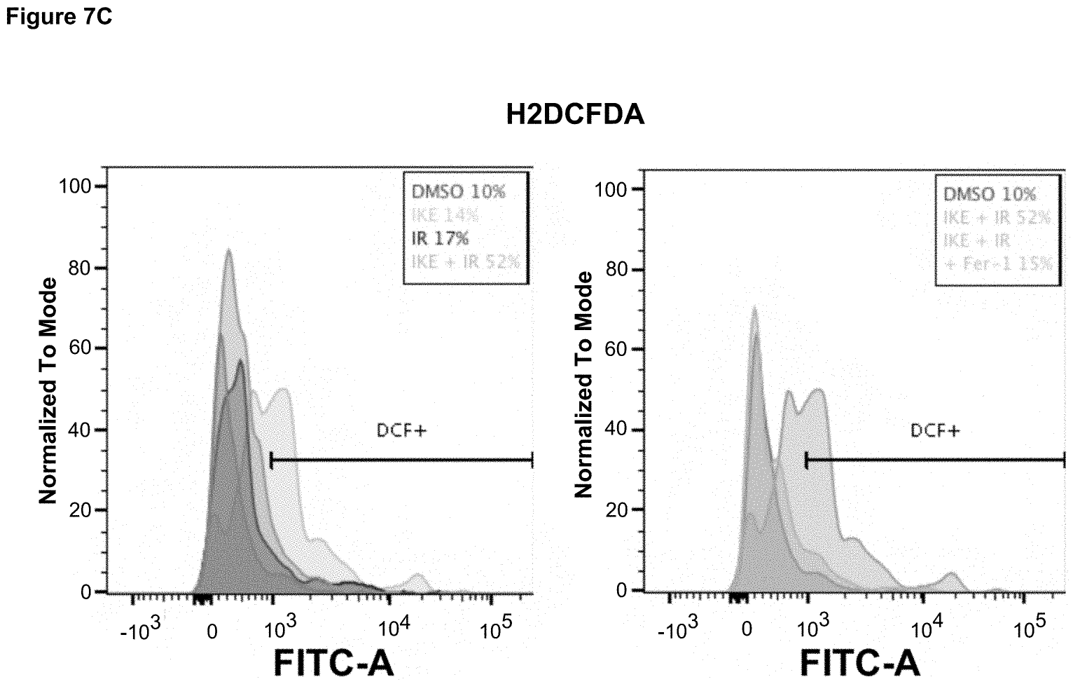

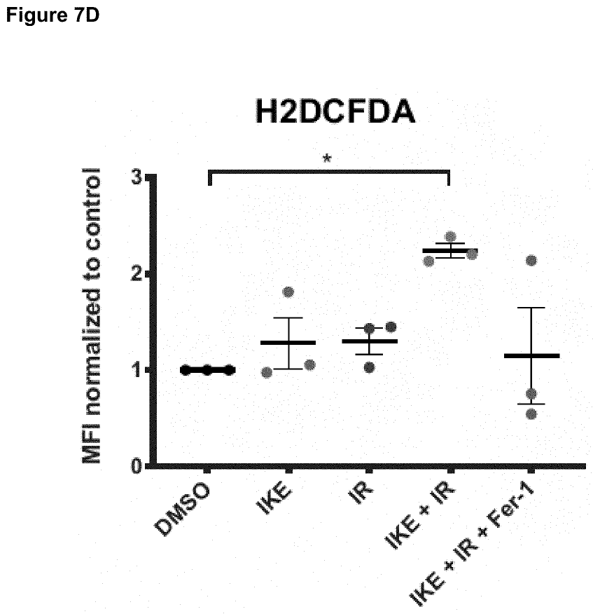

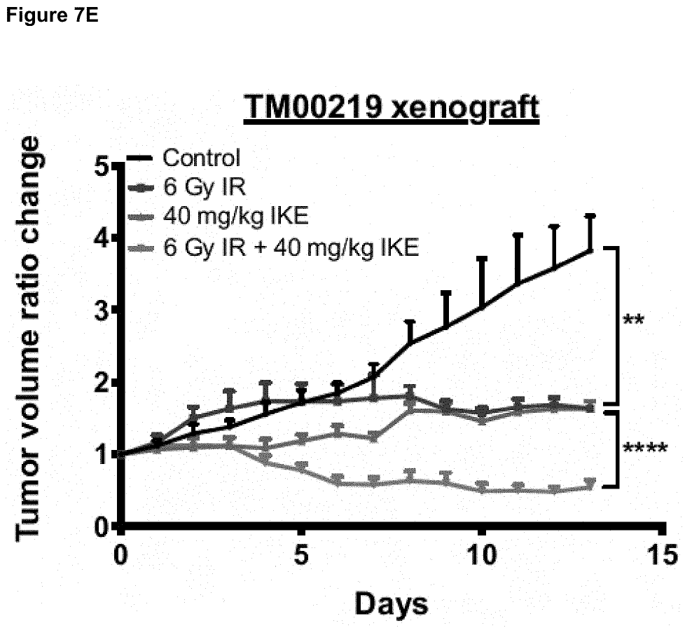

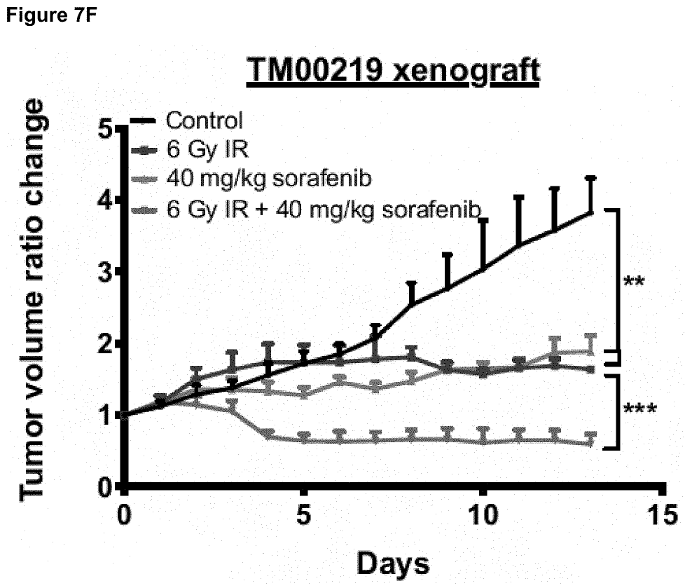

To determine if system x_{c}⁻ can potentially be an additional therapeutic target in tumors undergoing radiation therapy, the association between SLC7A11 expression and methylation and clinical outcomes was examined for all patients diagnosed with glioma in the Cancer Genome Atlas (TCGA) dataset (Brennan et al., 2013; Cancer Genome Atlas Research et al., 2015). Comparing patient groups with low (quartile 1) or high (quartile 4) levels of SLC7A11 RNA expression, we found that high expression of SLC7A11 RNA was associated with decreased overall survival (OS) and disease-free survival (DFS, p<0.001). Conversely, SLC7A11 DNA methylation was associated with improved OS and DFS (p<0.001) (FIGS. 7A and 12). Given that obtained data suggests that inhibition of system x_{c}⁻ sensitizes glioma cells to radiation-induced ferroptosis, it would also be expected that RNA expression and DNA methylation of SLC7A11 is preferentially important for patients treated with radiation over those who are not. In order to further determine whether survival outcomes were specific to radiation therapy, a subgroup analysis was conducted in the dataset, in which survival based on gene expression and methylation was stratified by receipt of radiation therapy. For patients who were not treated with radiotherapy, there was no association between survival and levels of SLC7A11 RNA expression or DNA methylation. However, in patients treated with radiation therapy, high SLC7A11 RNA expression was associated with decreased DFS (p<0.001), while high DNA methylation was associated with improved DFS (p<0.001). (FIG. 7B). Taken together, this data supports a role for SLC7A11 in treatment resistance of gliomas towards radiation, and suggests a potential benefit for system x_{c}⁻ inhibition with IKE or sorafenib during radiation treatment.

### Example 10

### IKE Combines with Radiation to Enhance Lipid Peroxidation in Ex Vivo Slice Cultures of Mouse and Human Gliomas

To further study the potential of combining small-molecule ferroptosis inducers and radiation for human therapeutic use, ex vivo samples of human glioma, which have been immediately cut from freshly resected tumors and grown as organotypic slice cultures, were used as previously described (Parker et al., 2017). De-identified patient information for these tumor samples are recorded in Table 4. The slices were then treated with 10 μM IKE, 2 Gy radiation, or a combination of both for 24 hours. Then, cells were dissociated and stained with H2DCFDA dye to measure formation of intracellular ROS using flow cytometry. A total of five human samples were tested. Of these, two glioblastomas did not demonstrate an increase in ROS generation following treatment with radiation, whereas three did respond to radiotherapy, including one high-grade oligodendroglioma and two astrocytomas. In the three responsive slice cultures, we also observed a significant enhancement of ROS generation with combination treatment when compared to control (FIGS. 7C and 7D). The ROS accumulation was also partially suppressible by co-treating with 10 μM ferrostatin-1, indicating that part of the generated ROS originates from lipid membranes. Tumor cell viability within slices was not assessed, as the number of tumor cells embedded in each slice cannot be normalized between slices. Taken together, these experiments show that certain human gliomas may be susceptible to a combination therapy of a ferroptosis inducer and radiation. However, a mixed response to radiation among all tumors tested suggests that more experiments are needed to better understand which types of glioma might be most sensitive to the proposed treatment regimen.

TABLE 4
Characteristics of human gliomas from which organotypic brain slice
cultures were derived.

| | | | |
|---|---|---|---|
| Tumor bank ID | Age | Sex | Diagnosis |

Positive response to radiation

| | | | |
|---|---|---|---|
| 6163 | 23 | M | Diffuse Astrocytoma, grade II |
| 6177 | 52 | M | Anaplastic Astrocytoma, grade III |
| 6181 | 32 | F | Anaplastic Oligodendroglioma, grade III |

Negative response to radiation

| | | | |
|---|---|---|---|
| 6186 | 66 | M | Glioblastoma, grade IV |
| 6193 | 67 | M | Glioblastoma, grade IV |

### Example 11

### IKE and Sorafenib Enhance Effects of Radiation to Inhibit Tumor Growth in a Patient-Derived Xenograft of Lung Adenocarcinoma

Finally, the effect of ferroptosis-inducing system x_{c}⁻ inhibitors, in combination with radiation, was tested on a patient-derived xenograft (PDX) model of lung adenocarcinoma, which is another type of cancer commonly treated with radiation therapy. NSG mice engrafted with a human lung adenocarcinoma tumor (TM00219) were evaluated. When the tumors reached an average volume of approximately 60 cubic millimeters, intraperitoneal (i.p.) injections of 40 mg/kg IKE, 40 mg/kg sorafenib, or vehicle were delivered daily for 14 days, as described above in the HT-1080 xenograft study. On day one of treatment, 0 or 6 Gy radiation was delivered to the tumor site using the SARRP. At the conclusion of the study, we observed significant tumor growth inhibition in the combination-treated group compared to all other groups (FIGS. 7E and 7F). In addition, treatment with radiation alone, IKE alone, or sorafenib alone also showed significant tumor control compared to the group treated with vehicle only. This experiment further demonstrates the applicability of this therapeutic strategy to patients whose cancers currently already receive radiation therapy as standard of care.

### Example 12

### Discussion

Mechanisms of cellular lethality from radiation have focused on the role of clustered DNA damage, in particular double-strand DNA breaks, in the nucleus. This genotoxicity leads to downstream effects such as apoptosis and mitotic catastrophe, which are thought to be the predominant mechanisms of cancer cell death following irradiation (Eriksson and Stigbrand, 2010). Nevertheless, some prior reports have highlighted the capacity for radiation to produce hydroxyl radicals and even lipid peroxidation in cell membranes (Shadyro et al., 2002; Walden et al., 1988). With the framework of ferroptosis, a lipid-peroxidation-based form of regulated cell death that can be modulated by a wide arsenal of pharmacological agents and metabolic interventions, it may now be possible to enhance the radiation-induced lipid damage response to kill tumors. This alternative mechanism may be especially effective in tumors with either intrinsic or acquired resistance to the genotoxic effects of radiation, such as those with increased capacity for DNA repair or a defective apoptosis pathway.

Intriguingly, the ferroptosis-inducing molecules erastin and sulfasalazine have been shown previously to potentiate radiation in models of glioma, melanoma, and breast cancer (Cobler et al., 2018; Nagane et al., 2018; Sleire et al., 2015). Both of these molecules belong to class I ferroptosis inducers, which inhibit system x_{c}⁻ and decreases glutathione synthesis. While these studies noted lowered levels of glutathione to be the cause of the compounds' radiosensitizing effect, the proposed mechanisms of synergy were proposed to be enhanced DNA damage and downstream apoptotic pathways. Our study is the first to link radiation to glutathione depletion resulting in lipid peroxidation, non-DNA damage, and subsequent ferroptotic cell death. These studies together also suggest that altered glutathione metabolism may have the ability to activate distinct cell death mechanisms.

While its ability to deplete glutathione is one likely mechanism by which radiation synergizes with ferroptosis inducers, other possibilities may be considered. Gamma rays and X-rays, the most clinically relevant types of radiation therapy, do not damage biological molecules directly, but rather through hydroxyl radical intermediates. Polyunsaturated fatty acids (PUFAs), which are oxidized in ferroptosis, are particularly sensitive to this type of damage given their ability to stabilize a free radical in the bis-allylic position through conjugation, and are the most sensitive lipid species to destruction when exposed to high dose radiation (Hammer and Wills, 1979). Evidence to support this hypothesis includes reports from two groups that treatment of several cancer cell lines, including astrocytomas and colorectal cancers, with PUFAs resulted in enhanced cell killing by radiation (Cai et al., 2014; Vartak et al., 1997). In addition, in a report identifying the role of ATM in iron metabolism and ferroptosis, the authors speculate that a radiation-induced increase of intracellular iron may be induced through ATM expression and that this increase may provide a further mechanism by which radiation can potentiate ferroptosis (Chen et al. 2019). Furthermore, it was recently reported that radiotherapy-activated ATM suppresses SLC7A11, triggering ferroptosis through decreased cysteine uptake and lipid peroxidation (Lang et al. 2019). Therefore, it is plausible that one of the ways in which glutathione depletion occurs in our models is due to ATM activation, although the authors agree with our view that many other mechanisms may be involved in this process. Additional studies are required to determine which of these mechanisms are the most relevant in diverse contexts.

While the upstream mechanism by which radiation oxidizes these lipids remains to be elucidated, our lipidomics data suggest that the effects of radiation on lipid species of treated cells as a whole overlap with those produced by a ferroptosis inducer such as IKE. In particular, the profound increase in lysophospholipids, a by-product of PUFA-phospholipid oxidation, has the potential to serve as a biomarker for ferroptosis induced by both radiation and IKE. For example, the levels of both LysoPI 18:1 and LysoPE 18:1 increased in both groups and showed strong interactions in the combination group, potentially acting as signatures of the synergistic effects between IKE and radiation.

The microbeam results we observed indicate that the interactions between radiation and the ferroptotic pathway occur primarily in the cytoplasm. At the same time, the lower threshold for cell killing by nuclear radiation compared to cytoplasmic radiation suggests that DNA damage is a major cell death modality of radiation. This is potentially because the charged particle radiation used for the microbeam experiments are known to have a higher Linear Energy Transfer (LET), causing more direct DNA damage when compared to gamma radiation used in cell culture experiments. Thus, the type of radiation may strongly impact the type of cell death activated, in addition to the tumor context. Overall, these findings offer a proof of concept for an alternative strategy for treating cancers that have evolved specific resistance mechanisms to DNA damage and repair. As ferroptosis inducers are optimized and developed as chemotherapeutic agents, these results suggest that they may be combined with radiation therapy in a variety of contexts. In particular, the use of this combination therapy in cancers that have undergone EMT is potentially promising, as they have been identified as a resistant state susceptible to GPX4 inhibition and ferroptotic cell death (Viswanathan et al., 2017). In these cases, the synergistic effects of this therapeutic strategy may allow lower doses of radiation to be delivered, therefore reducing adverse effects of radiation in healthy tissues.

Finally, using a combination of radiation and IKE or sorafenib, we showed that synergistic tumor cell killing through ferroptosis can be extended to patient-derived models of glioma and lung cancer. Given that these two types of cancer are routinely treated with radiation therapy, our findings potentially pave the way for the first clinical trial focused on ferroptosis as an alternative cell death pathway for tumor control in a therapeutically relevant cancer type.

In summary, the present disclosure provides that ferroptosis is a mechanism of radiation-induced cancer cell death, and that ferroptosis inducers act as radiosensitizers by potentiating the effects of radiation on cytoplasmic lipid peroxidation leading to cell death, in cell culture, xenograft mouse studies, and patient-derived xenografts and tumor slice cultures. These findings may open up new avenues of treatment for tumors that become resistant to conventional DNA damage and cell death pathways.

### DOCUMENTS

Brennan, C. W., Verhaak, R. G., McKenna, A., Campos, B., Noushmehr, H., Salama, S. R., Zheng, S., Chakravarty, D., Sanborn, J. Z., Berman, S. H., et al. (2013). The somatic genomic landscape of glioblastoma. Cell 155, 462-477.
Cai, F., Sorg, O., Granci, V., Lecumberri, E., Miralbell, R., Dupertuis, Y. M., and Pichard, C. (2014). Interaction of omega-3 polyunsaturated fatty acids with radiation therapy in two different colorectal cancer cell lines. Clin Nutr 33, 164-170.
Cancer Genome Atlas Research, N., Brat, D. J., Verhaak, R. G., Aldape, K. D., Yung, W. K., Salama, S. R., Cooper, L. A., Rheinbay, E., Miller, C. R., Vitucci, M., et al. (2015). Comprehensive, Integrative Genomic Analysis of Diffuse Lower-Grade Gliomas. N Engl J Med 372, 2481-2498.
Chaurasia, M., Bhatt, A. N., Das, A., Dwarakanath, B. S., and Sharma, K. (2016). Radiation-induced autophagy: mechanisms and consequences. Free Radic Res 50, 273-290.
Chen, P. H., Wu, J., Ding, C. C., Lin, C. C., Pan, S., Bossa, N., Xu, Y., Yang, W. H., Mathey-Prevot, B., and Chi, J. T. (2019) Kinome screen of ferroptosis reveals a novel role of ATM in regulating iron metabolism, Cell Death Differ., July 18. DOI: 10.1038/s41418-019-0393-7.
Cobler, L., Zhang, H., Suri, P., Park, C., and Timmerman, L. A. (2018). xCT inhibition sensitizes tumors to gamma-radiation via glutathione reduction. Oncotarget 9, 32280-32297.
Colles, S. M., and Chisolm, G. M. (2000). Lysophosphatidylcholine-induced cellular injury in cultured fibroblasts involves oxidative events. J Lipid Res 41, 1188-1198.
Delaney, G., Jacob, S., Featherstone, C., and Barton, M. (2005). The role of radiotherapy in cancer treatment: estimating optimal utilization from a review of evidence-based clinical guidelines. Cancer 104, 1129-1137.
Dixon, S. J., Lemberg, K. M., Lamprecht, M. R., Skouta, R., Zaitsev, E. M., Gleason, C. E., Patel, D. N., Bauer, A. J., Cantley, A. M., Yang, W. S., et al. (2012). Ferroptosis: an iron-dependent form of nonapoptotic cell death. Cell 149, 1060-1072.
Dixon, S. J., Patel, D. N., Welsch, M., Skouta, R., Lee, E. D., Hayano, M., Thomas, A. G., Gleason, C. E., Tatonetti, N. P., Slusher, B. S., et al. (2014). Pharmacological inhibition of cystine-glutamate exchange induces endoplasmic reticulum stress and ferroptosis. Elife 3, e02523.
Eriksson, D., and Stigbrand, T. (2010). Radiation-induced cell death mechanisms. Tumour Biol 31, 363-372.
Gao, M., Monian, P., Pan, Q., Zhang, W., Xiang, J., and Jiang, X. (2016) Ferroptosis is an autophagic cell death process. Cell Res. 26, 1021-1032.
Gaschler, M. M., Andia, A. A., Liu, H., Csuka, J. M., Hurlocker, B., Vaiana, C. A., Heindel, D. W., Zuckerman, D. S., Bos, P. H., Reznik, E., et al. (2018). FINO2 initiates ferroptosis through GPX4 inactivation and iron oxidation. Nat Chem Biol 14, 507-515.
Gerszten, P. C., Mendel, E., and Yamada, Y. (2009). Radiotherapy and radiosurgery for metastatic spine disease: what are the options, indications, and outcomes? Spine (Phila Pa. 1976) 34, S78-92.
Goldstein, M., and Kastan, M. B. (2015). The DNA damage response: implications for tumor responses to radiation and chemotherapy. Annu Rev Med 66, 129-143.
Hammer, C. T., and Wills, E. D. (1979). The effect of ionizing radiation on the fatty acid composition of natural fats and on lipid peroxide formation. Int J Radiat Biol Relat Stud Phys Chem Med 35, 323-332.
Hei, T. K., Wu, L. J., Liu, S. X., Vannais, D., Waldren, C. A., and Randers-Pehrson, G. (1997). Mutagenic effects of a single and an exact number of alpha particles in mammalian cells. Proc Natl Acad Sci USA 94, 3765-3770.
Kim, B. M., Hong, Y., Lee, S., Liu, P., Lim, J. H., Lee, Y. H., Lee, T. H., Chang, K. T., and Hong, Y. (2015). Therapeutic Implications for Overcoming Radiation Resistance in Cancer Therapy. Int J Mol Sci 16, 26880-26913.
Lang, X., Green, M. D., Wang, W., Yu, J., Choi, J. E., Jiang, L., Liao, P., Zhou, J., Zhang, Q., Dow, A., Saripalli, A. L., Kryczek, I., Wei, S., Szeliga, W., Vatan, L., Stone, E. M., Georgiou, G., Cieslik, M., Wahl, D. R., Morgan, M. A., Chinnaiyan, A. M., Lawrence, T. S., and Zou, W. (2019) Radiotherapy and immunotherapy promote tumoral lipid oxidation and ferroptosis via synergistic repression of SLC7A11. Cancer Discovery 9 (12), 1673-1685.
Larraufie, M. H., Yang, W. S., Jiang, E., Thomas, A. G., Slusher, B. S., and Stockwell, B. R. (2015). Incorporation of metabolically stable ketones into a small molecule probe to increase potency and water solubility. Bioorg Med Chem Lett 25, 4787-4792.
Liu, Z., Silke, J., and Hildebrand, J. M. (2018). Methods for Studying TNF-Mediated Necroptosis in Cultured Cells. Methods Mol Biol 1857, 53-61.
Lomax, M. E., Folkes, L. K., and O'Neill, P. (2013). Biological consequences of radiation-induced DNA damage: relevance to radiotherapy. Clin Oncol (R Coll Radiol) 25, 578-585.
Morgan, M. A., and Lawrence, T. S. (2015). Molecular Pathways: Overcoming Radiation Resistance by Targeting DNA Damage Response Pathways. Clin Cancer Res 21, 2898-2904.
Nagane, M., Kanai, E., Shibata, Y., Shimizu, T., Yoshioka, C., Maruo, T., and Yamashita, T. (2018). Sulfasalazine, an inhibitor of the cystine-glutamate antiporter, reduces DNA damage repair and enhances radiosensitivity in murine B16F10 melanoma. PLoS One 13, e0195151.
Nehs, M. A., Lin, C. I., Kozono, D. E., Whang, E. E., Cho, N. L., Zhu, K., Moalem, J., Moore, F. D., Jr., and Ruan, D. T. (2011). Necroptosis is a novel mechanism of radiation-induced cell death in anaplastic thyroid and adrenocortical cancers. Surgery 150, 1032-1039.
Parker, J. J., Lizarraga, M., Waziri, A., and Foshay, K. M. (2017). A Human Glioblastoma Organotypic Slice Culture Model for Study of Tumor Cell Migration and Patient-specific Effects of Anti-Invasive Drugs. J Vis Exp.
Pikman, Y., Alexe, G., Roti, G., Conway, A. S., Furman, A., Lee, E. S., Place, A. E., Kim, S., Saran, C., Modiste, R., et al. (2017). Synergistic Drug Combinations with a CDK4/6 Inhibitor in T-cell Acute Lymphoblastic Leukemia. Clin Cancer Res 23, 1012-1024.
Randers-Pehrson, G., Johnson, G. W., Marino, S. A., Xu, Y., Dymnikov, A. D., and Brenner, D. J. (2009). The Columbia University Sub-micron Charged Particle Beam. Nucl Instrum Methods Phys Res A 609, 294-299.
Shadyro, O. I., Yurkova, I. L., and Kisel, M. A. (2002). Radiation-induced peroxidation and fragmentation of lipids in a model membrane. Int J Radiat Biol 78, 211-217.
Skouta, R., Dixon, S. J., Wang, J., Dunn, D. E., Orman, M., Shimada, K., Rosenberg, P. A., Lo, D. C., Weinberg, J. M., Linkermann, A., et al. (2014). Ferrostatins inhibit oxidative lipid damage and cell death in diverse disease models. J Am Chem Soc 136, 4551-4556.
Sleire, L., Skeie, B. S., Netland, I. A., Forde, H. E., Dodoo, E., Selheim, F., Leiss, L., Heggdal, J. I., Pedersen, P. H., Wang, J., et al. (2015). Drug repurposing: sulfasalazine sensitizes gliomas to gamma knife radiosurgery by blocking cystine uptake through system Xc−, leading to glutathione depletion. Oncogene 34, 5951-5959.
Souhami, R. L., and Tobias, J. S. (2003). Cancer and its management, 4th edn (Malden, Mass.: Blackwell Science).
Stockwell, B. R., Friedmann Angeli, J. P., Bayir, H., Bush, A. I., Conrad, M., Dixon, S. J., Fulda, S., Gascon, S., Hatzios, S. K., Kagan, V. E., et al. (2017). Ferroptosis: A Regulated Cell Death Nexus Linking Metabolism, Redox Biology, and Disease. Cell 171, 273-285.
Tang, Z., Zeng, Q., Li, Y., Zhang, X., Ma, J., Suto, M. J., Xu, B., and Yi, N. (2017). Development of a radiosensitivity gene signature for patients with soft tissue sarcoma. Oncotarget 8, 27428-27439.
Vartak, S., Robbins, M. E., and Spector, A. A. (1997). Polyunsaturated fatty acids increase the sensitivity of 36610 rat astrocytoma cells to radiation-induced cell kill. Lipids 32, 283-292.
Viswanathan, V. S., Ryan, M. J., Dhruv, H. D., Gill, S., Eichhoff, O. M., Seashore-Ludlow, B., Kaffenberger, S. D., Eaton, J. K., Shimada, K., Aguirre, A. J., et al. (2017). Dependency of a therapy-resistant state of cancer cells on a lipid peroxidase pathway. Nature 547, 453-457.
Walden, T. L., Hughes, H. N., and SpringerLink (Online service) (1988). Prostaglandin and Lipid Metabolism in Radiation Injury (Boston, Mass.: Springer US,).
Willers, H., Azzoli, C. G., Santivasi, W. L., and Xia, F. (2013). Basic mechanisms of therapeutic resistance to radiation and chemotherapy in lung cancer. Cancer J 19, 200-207.
Wu, C. C., Chaudhary, K. R., Na, Y. H., Welch, D., Black, P. J., Sonabend, A. M., Canoll, P., Saenger, Y. M., Wang, T. J. C., Wuu, C. S., et al. (2017). Quality Assessment of Stereotactic Radiosurgery of a Melanoma Brain Metastases Model Using a Mouselike Phantom and the Small Animal Radiation Research Platform. Int J Radiat Oncol Biol Phys 99, 191-201.
Wu, L. J., Randers-Pehrson, G., Xu, A., Waldren, C. A., Geard, C. R., Yu, Z., and Hei, T. K. (1999). Targeted cytoplasmic irradiation with alpha particles induces mutations in mammalian cells. Proc Natl Acad Sci USA 96, 4959-4964.
Yagoda, N., von Rechenberg, M., Zaganjor, E., Bauer, A. J., Yang, W. S., Fridman, D. J., Wolpaw, A. J., Smukste, I., Peltier, J. M., Boniface, J. J., et al. (2007). RAS-RAF-MEK-dependent oxidative cell death involving voltage-dependent anion channels. Nature 447, 864-868.
Yamada, S., Kumazawa, S., Ishii, T., Nakayama, T., Itakura, K., Shibata, N., Kobayashi, M., Sakai, K., Osawa, T., and Uchida, K. (2001). Immunochemical detection of a lipofuscin-like fluorophore derived from malondialdehyde and lysine. J Lipid Res 42, 1187-1196.
Yang, W. S., and Stockwell, B. R. (2016). Ferroptosis: Death by Lipid Peroxidation. Trends Cell Biol 26, 165-176.
Yang, W. S., Kim, K. J., Gaschler, M. M., Patel, M., Shchepinov, M. S., and Stockwell, B. R. (2016). Peroxidation of polyunsaturated fatty acids by lipoxygenases drives ferroptosis. Proc Natl Acad Sci USA 113, E4966-4975.
Yang, W. S., SriRamaratnam, R., Welsch, M. E., Shimada, K., Skouta, R., Viswanathan, V. S., Cheah, J. H., Clemons, P. A., Shamji, A. F., Clish, C. B., et al. (2014). Regulation of ferroptotic cancer cell death by GPX4. Cell 156, 317-331.
Zhang, Y., Tan, H., Daniels, J. D., Zandkarimi, F., Liu, H., Brown, L. M., Uchida, K., O'Connor, O. A., and Stockwell, B. R. (2019). Imidazole Ketone Erastin Induces Ferroptosis and Slows Tumor Growth in a Mouse Lymphoma Model. Cell Chem Biol.
Zhou, H., Hong, M., Chai, Y., and Hei, T. K. (2009). Consequences of cytoplasmic irradiation: studies from microbeam. J Radiat Res 50 Suppl A, A59-65.
Zou, Y., Palte, M. J., Deik, A. A., Li, H., Eaton, J. K., Wang, W., Tseng, Y. Y., Deasy, R., Kost-Alimova, M., Dancik, V., et al. (2019). A GPX4-dependent cancer cell state underlies the clear-cell morphology and confers sensitivity to ferroptosis. Nat Commun 10, 1617.

All documents cited in this application are hereby incorporated by reference as if recited in full herein.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

What is claimed is:

1. A method for treating or ameliorating the effects of a cancer in a subject in need thereof, comprising co-administering to the subject i) a therapeutically effective amount of an agent that induces cell death and ii) a therapeutically effective amount of radiation.

2. The method of claim 1, wherein the cell death is selected from apoptosis, autophagy, necroptosis and ferroptosis.

3. The method of claim 1, wherein the cell death is ferroptosis.

4. The method of claim 1, wherein the agent is a ferroptosis inducer.

5. The method of claim 4, wherein the ferroptosis inducer is selected from the group consisting of erastin, imidazole ketone erastin (IKE), piperazine erastin (PE), sulfasalazine, sorafenib, Ras Synthetic Lethal 3 (RSL3), ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof.

6. The method of claim 1, the agent is selected from IKE, RSL3, sorafenib, and combinations thereof.

7. The method of claim 1, wherein the subject is a mammal.

8. The method of claim 7, wherein the mammal is selected from the group consisting of humans, primates, farm animals, and domestic animals.

9. The method of claim 1, wherein the subject is a human.

10. The method of claim 1, wherein the cancer is selected from the group consisting of sarcoma, renal cell carcinoma, diffuse large B-cell lymphoma, fibrosarcoma, glioma, uterine sarcoma, primary glioblastoma, lung cancer, non-small cell lung cancer, colorectal cancer, melanoma, prostate cancer, pancreatic cancer, brain cancer, breast cancer, colon cancer, liver cancer, leiomyosarcoma, lung adenocarcinoma, and hepatocyte-derived carcinoma.

11. The method of claim 1, wherein the cancer is resistant to radiation.

12. The method of claim 1, wherein the co-administration of the agent and radiation provides a synergistic effect compared to administration of either the agent or radiation alone.

13. A method for identifying and treating a subject with a cancer that is resistant to radiotherapy, comprising:
(a) administering radiotherapy to the subject;
(b) obtaining a biological sample from the subject;
(c) determining a SLC7A11 RNA expression level in the sample and comparing it to a predetermined reference;
(d) identifying the subject as having a cancer that is resistant to radiotherapy, if the SLC7A11 RNA expression level determined in step (c) is significantly higher than the reference; and
(e) treating the subject identified in step (d) as having a cancer that is resistant to radiotherapy by administering to the subject an effective amount of a radiosensitizer.

14. The method of claim 13, wherein the radiosensitizer is administered before, during and/or after radiotherapy.

15. The method of claim 13, wherein the cancer is selected from the group consisting of sarcoma, renal cell carcinoma, diffuse large B-cell lymphoma, fibrosarcoma, glioma, uterine sarcoma, primary glioblastoma, lung cancer, colorectal cancer, melanoma, prostate cancer, pancreatic cancer, brain cancer, breast cancer, colon cancer, liver cancer, leiomyosarcoma, lung adenocarcinoma, and hepatocyte-derived carcinoma.

16. The method of claim 13, wherein the cancer is a glioma.

17. The method of claim 13, wherein the subject is a human.

18. The method of claim 13, wherein the radiosensitizer is a ferroptosis inducer selected from the group consisting of erastin, imidazole ketone erastin (IKE), piperazine erastin (PE), sulfasalazine, sorafenib, Ras Synthetic Lethal 3 (RSL3), ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof.

19. The method of claim 13, wherein the radiosensitizer is selected from IKE, RSL3, sorafenib, and combinations thereof

20. A method for identifying and treating a subject with a cancer that is resistant to radiotherapy, comprising:
(a) administering radiotherapy to the subject;
(b) obtaining a biological sample from the subject;
(c) determining a SLC7A11 DNA methylation level in the sample and comparing it to a predetermined reference;
(d) identifying the subject as having a cancer that is resistant to radiotherapy, if the SLC7A11 DNA methylation level determined in step (c) is significantly lower than the reference; and
(e) treating the subject identified in step (d) as having a cancer that is resistant to radiotherapy by administering to the subject an effective amount of a radiosensitizer.

21. The method of claim 20, wherein the radiosensitizer is administered before, during and/or after radiotherapy.

22. The method of claim 20, wherein the cancer is selected from the group consisting of sarcoma, renal cell carcinoma, diffuse large B-cell lymphoma, fibrosarcoma, glioma, uterine sarcoma, primary glioblastoma, lung cancer, colorectal cancer, melanoma, prostate cancer, pancreatic cancer, brain cancer, breast cancer, colon cancer, liver cancer, leiomyosarcoma, lung adenocarcinoma, and hepatocyte-derived carcinoma.

23. The method of claim 20, wherein the cancer is a glioma.

24. The method of claim 20, wherein the subject is a human.

25. The method of claim 20, wherein the radiosensitizer is a ferroptosis inducer selected from the group consisting of erastin, imidazole ketone erastin (IKE), piperazine erastin (PE), sulfasalazine, sorafenib, Ras Synthetic Lethal 3 (RSL3), ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof.

26. The method of claim 20, wherein the radiosensitizer is selected from IKE, RSL3, sorafenib, and combinations thereof.

27. A method for enhancing the anti-tumor effect of radiation in a subject undergoing radiotherapy, comprising administering to the subject a therapeutically effective amount of a ferroptosis inducer.

28. The method of claim 27, wherein the ferroptosis inducer is selected from the group consisting of erastin, imidazole ketone erastin (IKE), piperazine erastin (PE), sulfasalazine, sorafenib, Ras Synthetic Lethal 3 (RSL3), ferroptosis inducer 56 (FIN56), caspase-independent lethal 56 (CIL56), deplete GPX4 protein, mevalonate-derived coenzyme Q₁₀, ferroptosis inducer endoperoxide (FINO₂), and combinations thereof.

29. The method of claim 27, wherein the ferroptosis inducer is selected from IKE, RSL3, sorafenib, and combinations thereof.

30. A method for enhancing the effect of radiation on a cancer cell, comprising contacting the cell with an effective amount of a ferroptosis inducer during radiation treatment.

31. The method of claim 30, wherein the ferroptosis inducer is selected from the group consisting of erastin, imidazole ketone erastin (IKE), piperazine erastin (PE), sulfasalazine, sorafenib, Ras Synthetic Lethal 3 (RSL3), ferroptosis inducer 56 (FIN56), caspase-independent lethal 56 (CIL56), deplete GPX4 protein, mevalonate-derived coenzyme Q₁₀, ferroptosis inducer endoperoxide (FIN02), and combinations thereof.

32. The method of claim 30, wherein the ferroptosis inducer is selected from IKE, RSL3, sorafenib, and combinations thereof.
